(19) United States
(12) Patent Application Publication
(10) Pub. No.: US 2022/0282330 A1
(43) Pub. Date: Sep. 8, 2022

(54) HLA-H IN MEDICINE AND DIAGNOSTICS

(71) Applicant: Intellexon GmbH, Pöcking (DE)

(72) Inventors: Wolfgang WÜRFEL, Odelzhausen (DE); Ralph Markus WIRTZ, Köln (DE); Christoph WINTERHALTER, Pöcking (DE); Franziska WÜRFEL, Odelzhausen (DE)

(21) Appl. No.: 17/625,063

(22) PCT Filed: Jul. 6, 2020
(86) PCT No.: PCT/EP2020/068989

§ 371 Date: Jan. 5, 2022

(30) Foreign Application Priority Data
Jul. 5, 2019 (EP) 19184729.2

(51) Int. Cl. C12Q 1/6881; A61K 31/7105; A61K 31/713; C07K 14/74

(52) U.S. Cl. CPC C12Q 1/6881; A61K 31/7105; A61K 31/713; C07K 14/70539; C12Q 2600/106; C12Q 2600/118; C12Q 2600/158

(57) ABSTRACT

The present invention relates to a nucleic acid molecule, a vector, a host cell, or a protein or peptide, or combinations thereof for use as an immunosuppressant, as a tumor vaccine or as a pregnancy promoter wherein (I) the nucleic acid molecule is (a) encoding a polypeptide comprising or consisting of the amino acid sequence of SEQ ID NO: 1; or (b) consisting of the nucleotide sequence of SEQ ID NO: 2; or (c) encoding a polypeptide which is at least 70%, preferably at least 80% identical, more preferably at least 90% identical, and most preferred at least 95% identical to the amino acid sequence of SEQ ID NO: 1; or (d) consisting of a nucleotide sequence which is at least 70% identical, preferably at least 80% identical, more preferably at least 90% identical, and most preferred at least 95% identical to the nucleotide sequence of SEQ ID NO: 2; or (e) consisting of a nucleotide sequence which is degenerate with respect to the nucleic acid molecule of (d); or (f) a fragment of the nucleic acid molecule of any one of (a) to (e), said fragment comprising at least 150 nucleotides, preferably at least 300 nucleotides, more preferably at least 450 nucleotides, and most preferably at least 600 nucleotides; or (g) corresponding to the nucleic acid molecule of any one of (a) to (f), wherein T is replaced by U; (II) the vector comprises the nucleic acid molecule of (I); (III) the host cell is transformed, transduced or transfected with the vector of (II); and (IV) the protein or peptide being encoded by the nucleic acid molecule of (I).

### RELATED PATENT APPLICATIONS

This patent application is a National Phase entry of, and claims priority to International Patent Application No. PCT/EP2020/068989 filed Jul. 6, 2020, entitled HLA-H IN MEDICINE AND DIAGNOSTICS, which claims priority to European Patent Application No. 19184729.2, filed Jul. 5, 2019. The entire content of the foregoing patent applications is incorporated herein by reference, including all text, tables and drawings.

### SEQUENCE LISTING

The instant application contains a Sequence Listing which has been submitted electronically in ASCII format and is hereby incorporated by reference in its entirety. Said ASCII copy, created on Dec. 23, 2021, is named “009848-0565964_sequence_listing” and is 16.5 KB in size.

The present invention relates to a nucleic acid molecule, a vector, a host cell, or a protein or peptide, or combinations thereof for use as an immunosuppressant, as a tumor vaccine or as a pregnancy promoter wherein (I) the nucleic acid molecule is (a) encoding a polypeptide comprising or consisting of the amino acid sequence of SEQ ID NO: 1; or (b) consisting of the nucleotide sequence of SEQ ID NO: 2; or (c) encoding a polypeptide which is at least 70%, preferably at least 80% identical, more preferably at least 90% identical, and most preferred at least 95% identical to the amino acid sequence of SEQ ID NO: 1; or (d) consisting of a nucleotide sequence which is at least 70% identical, preferably at least 80% identical, more preferably at least 90% identical, and most preferred at least 95% identical to the nucleotide sequence of SEQ ID NO: 2; or (e) consisting of a nucleotide sequence which is degenerate with respect to the nucleic acid molecule of (d); or (f) a fragment of the nucleic acid molecule of any one of (a) to (e), said fragment comprising at least 150 nucleotides, preferably at least 300 nucleotides, more preferably at least 450 nucleotides, and most preferably at least 600 nucleotides; or (g) corresponding to the nucleic acid molecule of any one of (a) to (f), wherein T is replaced by U; (II) the vector comprises the nucleic acid molecule of (I); (III) the host cell is transformed, transduced or transfected with the vector of (II); and (IV) the protein or peptide being encoded by the nucleic acid molecule of (I).

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The human leukocyte antigen (HLA) system or complex is a gene complex encoding the major histocompatibility complex (MHC) proteins in humans. These cell-surface proteins are responsible for the regulation of the immune system in humans. The HLA gene complex resides on a 3 Mbp stretch within chromosome 6p21. Genes in this complex are categorized into three basic groups: class I, class II, and class III.

Humans have three main MHC class I genes, known as HLA-A, HLA-B, and HLA-C. The proteins produced from these genes are present on the surface of almost all cells. On the cell surface, these proteins are bound to protein fragments (peptides) that have been exported from within the cell. MHC class I proteins display these peptides to the immune system. If the immune system recognizes the peptides as foreign (such as viral or bacterial peptides), it responds by triggering the infected cell to self-destruction.

There are six main MHC class II genes in humans: HLA-DPA1, HLA-DPB1, HLA-DQA1, HLA-DQB1, HLA-DRA, and HLA-DRB1. MHC class II genes provide instructions for making proteins that are present almost exclusively on the surface of certain immune system cells. Like MHC class I proteins, these proteins display peptides to the immune system.

The proteins produced from MHC class III genes have somewhat different functions; they are involved in inflammation and other immune system activities. The functions of some MHC genes are unknown.

HLA genes have many possible variations, allowing each person's immune system to react to a wide range of foreign invaders. Some HLA genes have hundreds of identified versions (alleles), each of which is given a particular number (such as HLA-B27). Closely related alleles are categorized together; for example, at least 40 very similar alleles are subtypes of HLA-B27. These subtypes are designated as HLA-B*2701 to HLA-B*2743.

More than 100 diseases have been associated with different alleles of HLA genes. For example, the HLA-B27 allele increases the risk of developing an inflammatory joint disease called ankylosing spondylitis. Many other disorders involving abnormal immune function and some forms of cancer have also been associated with specific HLA alleles. However, it is often unclear what role HLA genes play in the risk of developing these diseases.

Next to the three main MHC class I genes the non-classical MHC class I molecules HLA-E, HLA-F HLA-G are encoded by the HLA class I region. The overexpression of HLA-G, -E, and —F is a common finding across a variety of malignancies (Kochan et al., Oncoimmunology. 2013 Nov. 1; 2(11): e26491.). HLA-G and HLA-E were reported as being cancer biomarkers and also as being positively correlated with poor clinical outcome of cancer.

The HLA class I region was furthermore reported to include class I pseudogenes (Hughes, Mol Biol Evol. 1995 March; 12(2):247-58) as well as gene fragments. For instance, HLA-H, J, K and L are classified as class I pseudogenes and HLA-N, S and X are classified as gene fragments.

Hence, human leukocyte antigen (HLA) genes have a long research history as important targets in biomedical science and treatment. However, in view of the clinical importance of the HLA system there is still a need to focus research on the HLA genes and in particular to identify further targets for biomedical science and treatment based on the HLA system. This need is addressed by the present invention. In connection with the present invention it was surprisingly found that HLA-H is a target for the treatment and detection of diseases, in particular by either activating or inhibiting the activity of HLA-H.

The present invention therefore relates in a first aspect to a nucleic acid molecule, a vector, a host cell, or a protein or peptide, or combinations thereof for use as an immunosuppressant, as a tumor vaccine or as a pregnancy promoter wherein (I) the nucleic acid molecule is (a) encoding a polypeptide comprising or consisting of the amino acid sequence of SEQ ID NO: 1; or (b) consisting of the nucleotide sequence of SEQ ID NO: 2; or (c) encoding a polypeptide which is at least 70%, preferably at least 80% identical, more preferably at least 90% identical, and most preferred at least 95% identical to the amino acid sequence of SEQ ID NO: 1; or (d) consisting of a nucleotide sequence which is at least 70% identical, preferably at least 80% identical, more preferably at least 90% identical, and most preferred at least 95% identical to the nucleotide sequence of SEQ ID NO: 2; or (e) consisting of a nucleotide sequence which is degenerate with respect to the nucleic acid molecule of (d); or (f) a fragment of the nucleic acid molecule of any one of (a) to (e), said fragment comprising at least 150 nucleotides, preferably at least 300 nucleotides, more preferably at least 450 nucleotides, and most preferably at least 600 nucleotides; or (g) corresponding to the nucleic acid molecule of any one of (a) to (f), wherein T is replaced by U; (II) the vector comprises the nucleic acid molecule of (I); (III) the host cell is transformed, transduced or transfected with the vector of (II); and (IV) the protein or peptide being encoded by the nucleic acid molecule of (I).

The first aspect of the present invention likewise relates to a nucleic acid molecule, a vector, a host cell, or a protein or peptide, or combinations thereof for use as an immunosuppressant, as a tumor vaccine or as a pregnancy promoter wherein (I) the nucleic acid molecule (a) encodes a polypeptide comprising or consisting of the amino acid sequence of SEQ ID NO: 1 or 54; or (b) consists of the nucleotide sequence of SEQ ID NO: 2; or (c) encodes a polypeptide which is at least 70%, preferably at least 80% identical, more preferably at least 90% identical, and most preferred at least 95% identical to the amino acid sequence of SEQ ID NO: 1 or 54; or (d) consists of a nucleotide sequence which is at least 70% identical, preferably at least 80% identical, more preferably at least 90% identical, and most preferred at least 95% identical to the nucleotide sequence of SEQ ID NO: 2; or (e) consists of a nucleotide sequence which is degenerate with respect to the nucleic acid molecule of (d); or (f) is a fragment of the nucleic acid molecule of any one of (a) to (e), said fragment comprising at least 250 nucleotides, preferably at least 300 nucleotides, more preferably at least 450 nucleotides, and most preferably at least 600 nucleotides; or (g) corresponds to the nucleic acid molecule of any one of (a) to (f), wherein T is replaced by U; (II) the vector comprises the nucleic acid molecule of (I); (III) the host cell is transformed, transduced or transfected with the vector of (II); and (IV) the protein or peptide being encoded by the nucleic acid molecule of (I).

The term “nucleic acid molecule” in accordance with the present invention includes DNA, such as cDNA or double or single stranded genomic DNA and RNA. In this regard, “DNA” (deoxyribonucleic acid) means any chain or sequence of the chemical building blocks adenine (A), guanine (G), cytosine (C) and thymine (T), called nucleotide bases, that are linked together on a deoxyribose sugar backbone. DNA can have one strand of nucleotide bases, or two complimentary strands which may form a double helix structure. “RNA” (ribonucleic acid) means any chain or sequence of the chemical building blocks adenine (A), guanine (G), cytosine (C) and uracil (U), called nucleotide bases, that are linked together on a ribose sugar backbone. RNA typically has one strand of nucleotide bases, such as mRNA. Included are also single- and double-stranded hybrids molecules, i.e., DNA-DNA, DNA-RNA and RNA-RNA. The nucleic acid molecule may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Nucleic acid molecules, in the following also referred as polynucleotides, may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Further included are nucleic acid mimicking molecules known in the art such as synthetic or semi-synthetic derivatives of DNA or RNA and mixed polymers. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA), peptide nucleic acid (PNA) and locked nucleic acid (LNA) (see Braasch and Corey, Chem Biol 2001, 8: 1). LNA is an RNA derivative in which the ribose ring is constrained by a methylene linkage between the 2′-oxygen and the 4′-carbon. Also included are nucleic acids containing modified bases, for example thio-uracil, thio-guanine and fluoro-uracil. A nucleic acid molecule typically carries genetic information, including the information used by cellular machinery to make proteins and/or polypeptides. The nucleic acid molecule in accordance with the invention may additionally comprise promoters, enhancers, response elements, signal sequences, polyadenylation sequences, introns, 5′- and 3′-non-coding regions, and the like.

The nucleic acid molecule according to the invention encodes a polypeptide or fragment thereof which is derived from the HLA-H protein of SEQ ID NO: 1 or 54 which protein is encoded by SEQ ID NO: 2. It is therefore preferred that the nucleic acid molecule in accordance with the invention is genomic DNA or mRNA. In the case of mRNA, the nucleic acid molecule may in addition comprise a poly-A tail.

The term “protein” as used herein interchangeably with the term “polypeptide” describes linear molecular chains of amino acids, including single chain proteins or their fragments, containing at least 50 amino acids. The term “peptide” as used herein describes a group of molecules consisting of up to 49 amino acids, whereas the term “polypeptide” (also referred to as “protein”) as used herein describes a group of molecules consisting of at least 50 amino acids. The term “peptide” as used herein describes a group of molecules consisting with increased preference of at least 15 amino acids, at least 20 amino acids, at least 25 amino acids, and at least 40 amino acids. The group of peptides and polypeptides are referred to together by using the term “(poly)peptide”. (Poly)peptides may further form oligomers consisting of at least two identical or different molecules. The corresponding higher order structures of such multimers are, correspondingly, termed homo- or heterodimers, homo- or heterotrimers etc. The HLA-H protein of SEQ ID NO: 1 comprises cysteins at positions 93, 127, 229 and 285 and thus potential dimerization sites. Similarly, HLA-H protein of SEQ ID NO: 54 comprises cysteins at positions 89, 124, 225 and 281 and thus potential dimerization sites. Furthermore, peptidomimetics of such proteins/(poly)peptides where amino acid(s) and/or peptide bond(s) have been replaced by functional analogues are also encompassed by the invention. Such functional analogues include all known amino acids other than the 20 gene-encoded amino acids, such as selenocysteine. The terms “(poly)peptide” and “protein” also refer to naturally modified (poly)peptides and proteins where the modification is effected e.g. by glycosylation, acetylation, phosphorylation and similar modifications which are well known in the art.

In accordance with the present invention, the term “percent (%) sequence identity” describes the number of matches (“hits”) of identical nucleotides/amino acids of two or more aligned nucleic acid or amino acid sequences as compared to the number of nucleotides or amino acid residues making up the overall length of the template nucleic acid or amino acid sequences. In other terms, using an alignment, for two or more sequences or subsequences the percentage of amino acid residues or nucleotides that are the same (e.g. 70%, 75%, 80%, 85%, 90% or 95% identity) may be determined, when the (sub)sequences are compared and aligned for maximum correspondence over a window of comparison, or over a designated region as measured using a sequence comparison algorithm as known in the art, or when manually aligned and visually inspected. This definition also applies to the complement of any sequence to be aligned.

Nucleotide and amino acid sequence analysis and alignment in connection with the present invention are preferably carried out using the NCBI BLAST algorithm (Stephen F. Altschul, Thomas L. Madden, Alejandro A. Schäffer, Jinghui Zhang, Zheng Zhang, Webb Miller, and David J. Lipman (1997), Nucleic Acids Res. 25:3389-3402). BLAST can be used for nucleotide sequences (nucleotide BLAST) and amino acid sequences (protein BLAST). The skilled person is aware of additional suitable programs to align nucleic acid sequences.

As defined herein, sequence identities of at least 70% identical, preferably at least 80% identical, more preferably at least 90% identical, and most preferred at least 95% are envisaged by the invention. However, also envisaged by the invention are with increasing preference sequence identities of at least 97.5%, at least 98.5%, at least 99%, at least 99.5%, and at least 99.8%.

MHC class I molecules are generally comprised of two chains: a MHC alpha chain (heavy chain), and a beta2-microglobulin chain (light chain). Only the alpha chain spans the membrane. The alpha chain has three extracellular domains (being designated as alpha 1, 2 and 3 and with alpha 1 being at the N-terminus). It is believed that the alpha chain domains alpha 1 and alpha 3 of HLA-H predominately determine the immunosuppressive capability of HLA-H, wherein the domain alpha 3 is most important. It is of note that HLA-H comprises a truncated alpha 3 domain of only 13 amino acids whereas the alpha 3 domain of other HLA classes has about 93 amino acids. The nucleotide sequences of SEQ ID NOs 3 and 4 encode the domains alpha 1 and alpha 3 of HLA-H, respectively. The amino acid sequences of SEQ ID NOs 5 and 6 are the amino acid sequences of the domains alpha 1 and alpha 3 of HLA-H, respectively.

It is therefore preferred that the nucleotide sequences having at least 70% identity or any one of the preferred higher identities with the nucleotide sequence of SEQ ID NO: 2 comprise a nucleotide sequence being with increased preference at least 97.5%, at least 98.5%, at least 99%, at least 99.5%, at least 99.8%, and 100% identical to SEQ ID NO: 4. It is also preferred that the nucleotide sequences having at least 70% identity or any one of the preferred higher identities with the nucleotide sequence of SEQ ID NO: 2 encode an amino acid sequence being with increased preference at least 97.5%, at least 98.5%, at least 99%, at least 99.5%, at least 99.8%, and 100% identical to SEQ ID NO: 6. It is more preferred that the nucleotide sequences having at least 70% identity or any one of the preferred higher identities with the nucleotide sequence of SEQ ID NO: 2 comprise a nucleotide sequence being with increased preference at least 97.5%, at least 98.5%, at least 99%, at least 99.5%, at least 99.8%, and 100% identical to SEQ ID NO: 4 and/or comprise a nucleotide sequence being with increased preference at least 97.5%, at least 98.5%, at least 99%, at least 99.5%, at least 99.8%, and 100% identical to SEQ ID NO: 3. It is also more preferred that the nucleotide sequences having at least 70% identity or any one of the preferred higher identities with the nucleotide sequence of SEQ ID NO: 2 encode an amino acid sequence being with increased preference at least 97.5%, at least 98.5%, at least 99%, at least 99.5%, at least 99.8%, and 100% identical to SEQ ID NO: 6 and/or encode an amino acid sequence being with increased preference at least 97.5%, at least 98.5%, at least 99%, at least 99.5%, at least 99.8%, and 100% identical to SEQ ID NO: 5.

It is most preferred that the nucleotide sequences having at least 70% identity or any one of the preferred higher identities with the nucleotide sequence of SEQ ID NO: 2 comprise (i) a nucleotide sequence being with increased preference at least 97.5%, at least 98.5%, at least 99%, at least 99.5%, at least 99.8%, and 100% identical to SEQ ID NO: 4 and (ii) nucleotide sequence being with increased preference at least 97.5%, at least 98.5%, at least 99%, at least 99.5%, at least 99.8%, and 100% identical to SEQ ID NO: 3.

A particularly preferred example of an amino acid sequence sharing at least 95% identity with SEQ ID NO: 1 is the amino acid sequence of SEQ ID NO: 54. SEQ ID NO: 54 lacks the first 4 amino acids of SEQ ID NO: 1 but is otherwise identical to SEQ ID NO: 1. It has been found that the first four amino acids of SEQ ID NO: 1 are not important for the function of the HLA-H protein. Therefore SEQ ID NO: 54 may also replace or supplement SEQ ID NO: 1 as an alternative sequence of a HLA-H polypeptide in any of the embodiments as described herein.

The term “degenerate” in accordance with the present invention refers to the degeneracy of the genetic code. Degeneracy results because a triplet code designates 20 amino acids and a stop codon and because four bases exist which are utilized to encode genetic information, triplet codons are required to produce at least 21 different codes. The possible 4³ possibilities for bases in triplets give 64 possible codons, meaning that some degeneracy must exist. As a result, some amino acids are encoded by more than one triplet, i.e. by up to six. The degeneracy mostly arises from alterations in the third position in a triplet. This means that nucleic acid molecules having a different nucleotide sequence than that specified above, but still encoding the same polypeptide lie within the scope of the present invention. With regard to the first aspect of the invention, the skilled person thus understands that “(e) consisting of a nucleotide sequence which is degenerate with respect to the nucleic acid molecule of (d)” as recited in item (I)(e) designates a nucleic acid molecule which encodes the same amino acid sequence as the nucleic acid molecule according to item (I)(d). This amino acid sequence is either the amino acid sequence of SEQ ID NO: 1 or 54 or derived therefrom, said latter amino acid sequence being identical to SEQ ID NO: 1 or 54 at least to the extent as required and implied by the sequence identity values recited in item (I)(d) of the main embodiment.

Fragments of the nucleic acid molecule of any one of (I)(a) to (f) according the present first aspect of the invention comprise at least 150 nucleotides. In this regard, it is preferred with increasing preference that the fragments according the present invention are polynucleotides of at least 200, at least 250, at least 300, at least 350, at least 400, at least 450, at least 500, at least 550, at least 600, or at least 650 nucleotides and most preferred that the fragment is a fragment only lacking the 5′-ATP start codon and/or the 3′-TAG stop codon. Moreover, it is preferred that the fragment comprises a nucleotide sequence being with increased preference at least 97.5%, at least 98.5%, at least 99%, at least 99.5%, at least 99.8%, and 100% identical to SEQ ID NO: 4 or encodes an amino acid sequence being with increased preference at least 97.5%, at least 98.5%, at least 99%, at least 99.5%, at least 99.8%, and 100% identical to SEQ ID NO: 6. It is more preferred that the fragment comprises a nucleotide sequence being with increased preference at least 97.5%, at least 98.5%, at least 99%, at least 99.5%, at least 99.8%, and 100% identical to SEQ ID NO: 4 and/or a nucleotide sequence being with increased preference at least 97.5%, at least 98.5%, at least 99%, at least 99.5%, at least 99.8%, and 100% identical to SEQ ID NO: 3. Similarly, it is more preferred that the fragment encodes an amino acid sequence being with increased preference at least 97.5%, at least 98.5%, at least 99%, at least 99.5%, at least 99.8%, and 100% identical to SEQ ID NO: 6 and/or encodes an amino acid sequence being with increased preference at least 97.5%, at least 98.5%, at least 99%, at least 99.5%, at least 99.8%, and 100% identical to SEQ ID NO: 5. It is most preferred that the fragment comprises (i) a nucleotide sequence being with increased preference at least 97.5%, at least 98.5%, at least 99%, at least 99.5%, at least 99.8%, and 100% identical to SEQ ID NO: 4 and (ii) nucleotide sequence being with increased preference at least 97.5%, at least 98.5%, at least 99%, at least 99.5%, at least 99.8%, and 100% identical to SEQ ID NO: 3.

In accordance with a preferred embodiment of the first aspect of the invention the nucleic acid molecule is fused to a heterologous nucleotide sequence, preferably operably linked to a heterologous promoter.

The heterologous nucleotide sequence can either be directly or indirectly fused to the nucleic acid molecule in accordance with invention. In case of an indirect fusion preferably nucleotide sequences encoding a peptide linker are used for the fusion, such that a GS-linker (e.g. Gly-Gly-Gly-Gly-Ser)n (SEQ ID NO: 7), wherein n is 1 to 3).

As used herein, a heterologous nucleotide sequence is a sequence that cannot be found in nature fused to the nucleotide sequence of SEQ ID NO: 2. Noting that SEQ ID NO: 2 is from human, it is preferred that the heterologous nucleotide sequence is also derived from human.

Accordingly, a heterologous promoter is a promoter that cannot be found in nature operably linked to the nucleotide sequence of SEQ ID NO: 2. The heterologous promoter is preferably from human.

A promoter is a nucleic acid sequence that initiates transcription of a particular gene, said gene being in accordance with the invention derived from the HLA-H gene of SEQ ID NO: 2 or being SEQ ID NO: 2. In this connection “operably linked” shall mean that the heterologous promoter is fused to the nucleic acid molecule in accordance with invention, so that via the promoter the transcription of the nucleic acid molecule in accordance with the invention can be initiated, for example, in prokaryotes or eukaryotic cells. The heterologous promoter can be a constitutively active promoter, a tissue-specific or development-stage-specific promoter, an inducible promoter, or a synthetic promoter. Constitutive promoters direct expression in virtually all tissues and are largely, if not entirely, independent of environmental and developmental factors. As their expression is normally not conditioned by endogenous factors, constitutive promoters are usually active across species and even across kingdoms. Tissue-specific or development-stage-specific promoters direct the expression of a gene in specific tissue(s) or at certain stages of development. The activity of inducible promoters is induced by the presence or absence of biotic or abiotic factors. Inducible promoters are a very powerful tool in genetic engineering because the expression of genes operably linked to them can be turned on or off as needed. Synthetic promoters are constructed by bringing together the primary elements of a promoter region from diverse origins.

Non-limiting examples of heterologous promoters which are used in the art in order to express genes heterologously are SV40, CMV, HSV, UBC, EF1A, PGK, Vlambda1, RSV and CAGG (for mammalian systems); COPIA and ACT5C (for Drosophila systems) and GAL1, GAL10, GAL7, GAL2 (for yeast systems) and can also be employed in connection with the present invention.

Alternatively or in addition, the heterologous nucleic acid sequence may be a coding sequence such that the nucleic acid sequence of the invention gives rise to a fusion protein. Such fusion proteins are discussed in more detail herein below.

If the nucleic acid molecule is not fused to a heterologous promoter, then for expression purposes it is fused to its own promoter.

The term “vector” in accordance with the invention means preferably a plasmid, cosmid, virus, bacteriophage or another vector used e.g. conventionally in genetic engineering which carries the nucleic acid molecule in accordance with invention. The nucleic acid molecule in accordance with the invention may, for example, be inserted into several commercially available vectors. Non-limiting examples include prokaryotic plasmid vectors, such as of the pUC-series, pBluescript (Stratagene), the pET-series of expression vectors (Novagen) or pCRTOPO (Invitrogen) and vectors compatible with an expression in mammalian cells like pREP (Invitrogen), pcDNA3 (Invitrogen), pCEP4 (Invitrogen), pMC1 neo (Stratagene), pXT1 (Stratagene), pSG5 (Stratagene), EBO-pSV2neo, pBPV-1, pdBPVMMTneo, pRSVgpt, pRSVneo, pSV2-dhfr, pIZD35, pLXIN, pSIR (Clontech), pIRES-EGFP (Clontech), pEAK-10 (Edge Biosystems) pTriEx-Hygro (Novagen) and pCINeo (Promega). Examples for plasmid vectors suitable for Pichia pastoris comprise e.g. the plasmids pAO815, pPIC9K and pPIC3.5K (all Invitrogen).

The nucleic acid molecules inserted into the vector can e.g. be synthesized by standard methods, or isolated from natural sources. Ligation of the coding sequences to transcriptional regulatory elements and/or to other amino acid encoding sequences can also be carried out using established methods. Transcriptional regulatory elements (parts of an expression cassette) ensuring expression in prokaryotes or eukaryotic cells are well known to those skilled in the art. These elements comprise regulatory sequences ensuring the initiation of transcription (e. g., translation initiation codon, promoters, such as naturally-associated or heterologous promoters and/or insulators; see above), internal ribosomal entry sites (IRES) (Owens, Proc. Natl. Acad. Sci. USA 98 (2001), 1471-1476) and optionally poly-A signals ensuring termination of transcription and stabilization of the transcript. Additional regulatory elements may include transcriptional as well as translational enhancers. Preferably, the polynucleotide encoding the polypeptide/protein or fusion protein in accordance with the invention is operatively linked to such expression control sequences allowing expression in prokaryotes or eukaryotic cells. The vector may further comprise nucleic acid sequences encoding secretion signals as further regulatory elements. Such sequences are well known to the person skilled in the art. Furthermore, depending on the expression system used, leader sequences capable of directing the expressed polypeptide to a cellular compartment may be added to the coding sequence of the polynucleotide of the invention. Such leader sequences are well known in the art.

Furthermore, it is preferred that the vector comprises a selectable marker. Examples of selectable markers include genes encoding resistance to neomycin, ampicillin, hygromycine, and kanamycin. Specifically-designed vectors allow the shuttling of DNA between different hosts, such as bacteria-fungal cells or bacteria-animal cells (e. g. the Gateway system available at Invitrogen). An expression vector according to this invention is capable of directing the replication, and the expression, of the polynucleotide and encoded peptide or fusion protein of this invention. Apart from introduction via vectors such as phage vectors or viral vectors (e.g. adenoviral, retroviral), the nucleic acid molecules as described herein above may be designed for direct introduction or for introduction via liposomes into a cell. Additionally, baculoviral systems or systems based on vaccinia virus or Semliki Forest virus can be used as eukaryotic expression systems for the nucleic acid molecules of the invention.

The term “host cell” means any cell of any organism that is selected, modified, transformed, grown, or used or manipulated in any way, for the production of the protein or peptide or fusion protein in accordance with the invention by the cell.

The host cell of the invention is typically produced by introducing the nucleic acid molecule or vector(s) of the invention into the host cell which upon its/their presence mediates the expression of the nucleic acid molecule in accordance with the invention encoding the protein or peptide or fusion protein in accordance with the invention. The host from which the host cell is derived or isolated may be any prokaryote or eukaryotic cell or organism, preferably with the exception of human embryonic stem cells that have been derived directly by destruction of a human embryo.

Suitable prokaryotes (bacteria) useful as hosts for the invention are, for example, those generally used for cloning and/or expression like E. coli (e.g., E. coli strains BL21, HB101, DH5a, XL1 Blue, Y1090 and JM101), Salmonella typhimurium, Serratia marcescens, Burkholderia glumae, Pseudomonas putida, Pseudomonas fluorescens, Pseudomonas stutzeri, Streptomyces lividans, Lactococcus lactis, Mycobacterium smegmatis, Streptomyces coelicolor or Bacillus subtilis. Appropriate culture mediums and conditions for the above-described host cells are well known in the art.

A suitable eukaryotic host cell may be a vertebrate cell, an insect cell, a fungal/yeast cell, a nematode cell or a plant cell. The fungal/yeast cell may a Saccharomyces cerevisiae cell, Pichia pastoris cell or an Aspergillus cell. Preferred examples for host cell to be genetically engineered with the nucleic acid molecule or the vector(s) of the invention is a cell of yeast, E. coli and/or a species of the genus Bacillus (e.g., B. subtilis). In one preferred embodiment the host cell is a yeast cell (e.g. S. cerevisiae).

In a different preferred embodiment the host cell is a mammalian host cell, such as a Chinese Hamster Ovary (CHO) cell, mouse myeloma lymphoblastoid, human embryonic kidney cell (HEK-293), human embryonic retinal cell (Crucell's Per.C6), or human amniocyte cell (Glycotope and CEVEC). The cells are frequently used in the art to produce recombinant proteins. CHO cells are the most commonly used mammalian host cells for industrial production of recombinant protein therapeutics for humans.

The terms “protein” and “peptide” and preferred embodiments thereof have been defined herein above in connection with the first aspect of the invention. These definitions and preferred embodiments apply mutatis mutandis to the second aspect of the invention. The peptide in accordance with the invention is preferably at least 80%, preferably at least 90% and most preferably at least 95% identical to a subsequence of SEQ ID NO: 1 or 54.

The protein or peptide in accordance with the invention may be generated by molecular cloning techniques well known in the art. Recombinant expression can be accomplished, for example, by using vectors and host cells as described herein above.

According to a preferred embodiment, the protein or peptide in accordance with the invention is a fusion protein.

A “fusion protein” according to the present invention contains at least one additional heterologous amino acid sequence. Often, but not necessarily, these additional sequences will be located at the N- or C-terminal end of the (poly)peptide. It may e.g. be convenient to initially express the polypeptide as a fusion protein from which the additional amino acid residues can be removed, e.g. by a proteinase capable of specifically trimming the fusion protein and releasing the (poly)peptide of the present invention. The amino acid sequence compound can either be directly or indirectly fused to the nucleic acid molecule in accordance with invention. In case of an indirect fusion generally a peptide linker may be used for the fusion, such that a GS-linker (e.g. Gly-Gly-Gly-Gly-Ser)n (SEQ ID NO: 7), wherein n is 1 to 3).

Those at least one additional heterologous amino acid sequence of said fusion proteins includes amino acid sequences which confer desired properties such as modified/enhanced stability, modified/enhanced solubility and/or the ability of targeting one or more specific cell types. For example, fusion proteins with antibodies. The term antibody is further defined herein below and inter alia comprises antibody fragments and derivatives. The antibody may be, for example, specific for cell surface markers or may be an antigen-recognizing fragment of said antibodies. The protein or peptide in accordance with the invention can be fused to the N-terminus or C-terminus of the light and/or heavy chain(s) of an antibody. The protein or peptide in accordance with the invention is preferably fused to the N-terminus of the light and/or heavy chain(s) of an antibody, so that the Fc part of the antibody is free to bind to Fc-receptors.

The fusion protein may also comprise protein domains known to function in signal transduction and/or known to be involved in protein-protein interaction. Examples for such domains are Ankyrin repeats; arm, Bcl-homology, Bromo, CARD, CH, Chr, C1, C2, DD, DED, DH, EFh, ENTH, F-box, FHA, FYVE, GEL, GYF, hect, LIM, MH2, PDZ, PB1, PH, PTB, PX, RGS, RING, SAM, SC, SH2, SH3, SOCS, START, TIR, TPR, TRAF, tsnare, Tubby, UBA, VHS, W, WW, and 14-3-3 domains. Further information about these and other protein domains is available from the databases InterPro (http://www.ebi.ac.uk/interpro/, Mulder et al., 2003, Nucl. Acids. Res. 31: 315-318), Pfam (http://www.sanger.ac.uk/Software/Pfam/, Bateman et al., 2002, Nucleic Acids Research 30(1): 276-280) and SMART (http://smart.embl-heidelberg.de/, Letunic et al., 2002, Nucleic Acids Res. 30(1), 242-244).

The at least one additional heterologous amino acid sequence of the fusion protein according to the present invention may comprise or consist of (a) a cytokine, (b) a chemokine, (c) a pro-coagulant factor, (d) a proteinaceous toxic compound, and/or (e) an enzyme for pro-drug activation.

The cytokine is preferably selected from the group consisting of IL-2, IL-12, TNF-alpha, IFN alpha, IFN beta, IFN gamma, IL-10, IL-15, IL-24, GM-CSF, IL-3, IL-4, IL-5, IL-6, IL-7, IL-9, IL-11, IL-13, LIF, CD80, B70, TNF beta, LT-beta, CD-40 ligand, Fas-ligand, TGF-beta, IL-1 alpha and IL-1beta. As it is well-known in the art, cytokines may favour a pro-inflammatory or an anti-inflammatory response of the immune system. Thus, depending on the disease to be treated either fusion proteins with a pro-inflammatory or an anti-inflammatory cytokine may be favored. For example, for the treatment of inflammatory diseases in general fusion constructs comprising anti-inflammatory cytokines are preferred, whereas for the treatment of cancer in general fusion constructs comprising pro-inflammatory cytokines are preferred.

The chemokine is preferably selected from the group consisting of IL-8, GRO alpha, GRO beta, GRO gamma, ENA-78, LDGF-PBP, GCP-2, PF4, Mig, IP-10, SDF-1alpha/beta, BUNZO/STRC33, I-TAC, BLC/BCA-1, MIP-1alpha, MIP-1 beta, MDC, TECK, TARC, RANTES, HCC-1, HCC-4, DC-CK1, MIP-3 alpha, MIP-3 beta, MCP-1-5, eotaxin, Eotaxin-2, 1-309, MPIF-1, 6Ckine, CTACK, MEC, lymphotactin and fractalkine. The major role of chemokines is to act as a chemoattractant to guide the migration of cells. Cells that are attracted by chemokines follow a signal of increasing chemokine concentration towards the source of the chemokine. It follows that within the fusion protein the chemokin can be used to guide the migration of the protein or peptide in accordance with the invention, e.g. to a specific cells type or body site.

The pro-coagulant factor is preferably a tissue factor. A pro-coagulant factor promoting the process by which blood changes from a liquid to a gel, forming a blood clot. Pro-coagulant factors may, for example, aid in wound healing.

The proteinaceous toxic compound is preferably Ricin-A chain, modeccin, truncated Pseudomonas exotoxin A, diphtheria toxin and recombinant gelonin. Toxic compounds can have a toxic effect on a whole organism as well as on a substructure of the organism, such as a particular cell type. Toxic compounds are frequently used in the treatment of tumors. Tumor cells generally grow faster than normal body cells, so that they preferentially accumulate toxic compounds and in higher amounts.

The enzyme for pro-drug activation is preferably an enzyme selected from the group consisting of carboxy-peptidases, glucuronidases and glucosidases. Among the broad array of genes that have been evaluated for tumor therapy, those encoding pro-drug activation enzymes are especially appealing as they directly complement ongoing clinical chemotherapeutic regimes. These enzymes can activate prodrugs that have low inherent toxicity using both bacterial and yeast enzymes, or enhance prodrug activation by mammalian enzymes.

In accordance with a preferred embodiment, the protein or peptide is fused to a heterologous non-proteinaceous compound.

As used herein, a heterologous compound is a compound that cannot be found in nature fused to the amino acid sequence of SEQ ID NO: 1 or 54.

The heterologous non-proteinaceous compound can either be directly or indirectly fused to the nucleic acid molecule in accordance with invention. For, example chemical linker may be used. Chemical linkers may contain diverse functional groups, such as primary amines, sulfhydryls, acids, alcohols and bromides. Many of our crosslinkers are functionalized with maleimide (sulfhydral reactive) and succinimidyl ester (NHS) or isothiocyanate (ITC) groups that react with amines.

The heterologous non-proteinaceous compound is preferably a pharmaceutically active compound or diagnostically active compound. The pharmaceutically active compound or diagnostically active compound is preferably selected from the group consisting of (a) a fluorescent dye, (b) a photosensitizer, (c) a radionuclide, (d) a contrast agent for medical imaging, (e) a toxic compound, or (f) an ACE inhibitor, a Renin inhibitor, an ADH inhibitor, an Aldosteron inhibitor, an Angiotensin receptor blocker, a TSH-receptor, a LH-/HCG-receptor, an oestrogen receptor, a progesterone receptor, an androgen receptor, a GnRH-receptor, a GH (growth hormone) receptor, or a receptor for IGF-I or IGF-II.

The fluorescent dye is preferably a component selected from Alexa Fluor or Cy dyes.

The photosensitizer is preferably phototoxic red fluorescent protein KillerRed or haematoporphyrin.

The radionuclide is preferably either selected from the group of gamma-emitting isotopes, more preferably ^{99m}Tc, ¹²³I, ¹¹¹In, and/or from the group of positron emitters, more preferably ¹⁸F, ⁶⁴Cu, ⁶⁸Ga, ⁸⁶Y, ¹²⁴I, and/or from the group of beta-emitter, more preferably ¹³¹I, ⁹⁰Y, ¹⁷⁷Lu, ⁶⁷Cu, ⁹⁰Sr, or from the group of alpha-emitter, preferably ²¹³Bi, ²¹¹At.

A contrast agent as used herein is a substance used to enhance the contrast of structures or fluids within the body in medical imaging. Common contrast agents work based on X-ray attenuation and magnetic resonance signal enhancement.

The toxic compound is preferably a small organic compound, more preferably a toxic compound selected from the group consisting of calicheamicin, maytansinoid, neocarzinostatin, esperamicin, dynemicin, kedarcidin, maduropeptin, doxorubicin, daunorubicin, and auristatin. In contrast to the herein above described proteinaceous toxic compound these toxic compounds are non-proteinaceous.

The nucleic acid molecule, vector, host cell, or protein or peptide, or combinations in accordance with the invention thereof may be formulated as a pharmaceutical composition. In accordance with the present invention, the term “pharmaceutical composition” relates to a composition for administration to a patient, preferably a human patient. The pharmaceutical composition of the invention comprises the compounds recited above. It may, optionally, comprise further molecules capable of altering the characteristics of the compounds of the invention thereby, for example, stabilizing, modulating and/or activating their function. The composition may be in solid, liquid or gaseous form and may be, inter alia, in the form of (a) powder(s), (a) tablet(s), (a) solution(s) or (an) aerosol(s). The pharmaceutical composition of the present invention may, optionally and additionally, comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, organic solvents including DMSO etc. Compositions comprising such carriers can be formulated by well known conventional methods. These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgement of the ordinary clinician or physician. Generally, the regimen as a regular administration of the pharmaceutical composition should be in the range of 1 μg to 5 g units per day. However, a more preferred dosage might be in the range of 0.01 mg to 100 mg, even more preferably 0.01 mg to 50 mg and most preferably 0.01 mg to 10 mg per day. Furthermore, if for example said compound is an iRNA agent, such as an siRNA, the total pharmaceutically effective amount of pharmaceutical composition administered will typically be less than about 75 mg per kg of body weight, such as for example less than about 70, 60, 50, 40, 30, 20, 10, 5, 2, 1, 0.5, 0.1, 0.05, 0.01, 0.005, 0.001, or 0.0005 mg per kg of body weight. More preferably, the amount will be less than 2000 nmol of iRNA agent (e.g., about 4.4×10¹⁶ copies) per kg of body weight, such as for example less than 1500, 750, 300, 150, 75, 15, 7.5, 1.5, 0.75, 0.15, 0.075, 0.015, 0.0075, 0.0015, 0.00075 or 0.00015 nmol of iRNA agent per kg of body weight. The length of treatment needed to observe changes and the interval following treatment for responses to occur vary depending on the desired effect. The particular amounts may be determined by conventional tests which are well known to the person skilled in the art.

An immunosuppressant is a drug being capable of suppressing the immune response. They can be used in immunosuppressive therapy, for example, to (i) prevent the rejection of transplanted organs and tissues (e.g., bone marrow, heart, kidney, liver), (ii) treat autoimmune diseases or diseases that are most likely of autoimmune origin (e.g., rheumatoid arthritis, multiple sclerosis, myasthenia gravis, psoriasis, vitiligo, systemic lupus erythematosus, sarcoidosis, focal segmental glomerulosclerosis, Crohn's disease, Behcet's disease, pemphigus, sclerodermia and ulcerative colitis), and/or (iii) treat non-autoimmune inflammatory diseases (e.g., long term allergic asthma control and ankylosing spondylitis).

A tumor vaccine can either be used to treat an existing tumor or to prevent the development of a tumor. Vaccines that treat existing cancer are also known as therapeutic cancer vaccines. The vaccines may be “autologous”, i.e. being prepared from samples taken from the patient, and are specific to that patient. The approach of cancer vaccination is generally to separate proteins from cancer cells and immunize patients against those proteins as antigens, with the aim of stimulating the immune system to kill the cancer cells. The antigen is in accordance with the present invention derived from a HLA-H protein/peptide.

Accordingly, the present invention also relates to a method for the preparation of a tumor vaccine comprising admixing the nucleic acid molecule, the vector, the host cell, the protein or peptide, the binding molecule, preferably the inhibitor in accordance with the invention or combinations thereof with at least one pharmaceutically acceptable excipient, carrier and/or diluents.

A pregnancy promoter is a compound increasing the likelihood to become pregnant and in particular the likelihood of embryo implantation. Implantation is the stage of pregnancy at which the already fertilized egg adheres to the wall of the uterus. It is by this adhesion that the embryo receives oxygen and nutrients from the mother to be able to grow. In humans, adhesion and implantation of a fertilized ovum is most likely to occur around 5 to 6 days after ovulation. Implantation failure is considered to be caused by inadequate uterine receptivity in two-thirds of cases, and by problems with the embryo itself in the other third. This is also dependent on the age of the mother. Inadequate uterine receptivity is more frequent in younger mothers while problems with the embryo itself (e.g. chromosomal aberrations) are more frequent in older mothers (in particular above the age of 35 years). Inadequate uterine receptivity may be caused by abnormal cytokine and hormonal signaling as well as epigenetic alterations. Recurrent implantation failure is a cause of female infertility. Therefore, pregnancy rates can be improved by optimizing endometrial receptivity for implantation.

The nucleic acid molecule, the vector, the host cell, the protein or peptide, the binding molecule, preferably the inhibitor in accordance with the invention or combinations thereof can thus be used, for example, in in vitro fertilization, wherein the oocyte is cultured in the presence of the nucleic acid molecule, the vector, the host cell, the protein or peptide, the binding molecule, preferably the inhibitor in accordance with the invention or combinations thereof before it is fertilized and implanted into the mother.

The detection of HLA-H expression in tissue samples from cancer patients is shown in the appended examples. In more detail, in Examples 1 and 2 HLA-H expression in bladder cancer patients is shown, in Example 3 in bladder cancer patients before and after chemotherapy and in Example 4 in ovarian cancer patients before and after chemotherapy. Examples 2 to 4 show that high level of HLA-H expression is associated with an adverse outcome, e.g. a low survival rate upon checkpoint therapy or chemotherapy resistance. Moreover, it is shown in Example 4 that an increase of HLA-H expression is positively associated with a higher tumor stage. It thus can be safely assumed that HLA-H expression helps the tumor to escape the immune system. This in turn shows that HLA-H acts as an immunosuppressant.

This body of evidence shows that HLA-H is not a pseudogene but is in fact functional gene encoding a protein. In this respect reference is made to the pseudogene HLA-H gene entry of the database GeneCards (GC06P032554). The database entry mentions the amino acid sequence UniPortKB: P01893 and cautions that the protein could be the product of a pseudogene and characterizes the protein as “putative”. The experimental data herein unexpectedly revealed that HLA-H is not a pseudogene but in fact encodes a functional protein. Even more unexpectedly this functional protein is not the amino acid sequence UniPortKB: P01893 but the amino acid sequence of SEQ ID NO: 1.

The amino acid sequence UniPortKB: P01893 is based on a wrongly assumed open reading frame. For this reason SEQ ID NOs 1 and 54 as provided herein only share about 90% sequence identity with a subpart of UniPortKB: P01893. Yet further, UniPortKB: P01893 comprises a HLA transmembrane domain and the correct HLA-H as disclosed herein does not. The putative HLA-H UniPortKB: P01893—just as HLA-G—is membrane-bound while it was unexpectedly found that HLA-H is in fact a soluble HLA. It was not obvious from the prior art that the available sequences of the HLA-H pseudogenes and the putative HLA-H proteins as comprised in the public gene and protein databases are wrong, let alone that SEQ ID NOs 1 and 2 are the correct sequences.

The above-discussed data in the examples also renders it at least plausible that the vaccination of a tumor patient with HLA-H helps to suppress or abrogate the escape of the tumor from the immune system via HLA-H expression. Hence, the nucleic acid molecule, the vector, the host cell, or the protein or peptide of the invention or combinations can be used as a as an immunosuppressant or as a tumor vaccine. The nucleic acid molecule is preferably a nucleic acid molecule of item (g) of the first aspect. While WO 2018/140525 envisions the use of an HLA-H antibody for the treatment of cancer, WO 2018/140525 does not disclose any HLA-H, let alone the correct HLA-H sequences of SEQ ID NOs 1 and 2 as provided herein. Similarly, WO 2018/183921 refers to a long list of potential novel immunotherapy targets, wherein HLA-H is among this list. Again, no HLA-H sequences are disclosed.

Yet further, it is assumed that the nucleic acid molecule, the vector, the host cell, the protein or peptide, or combinations thereof can be used as pregnancy promoter, in particular for optimizing endometrial receptivity for implantation. The nucleic acid molecule is preferably a nucleic acid molecule of item (g) of the first aspect. This is because HLA-G is thought to play a key role in implantation by modulating cytokine secretion to control trophopblastic cell invasion and to maintain a local immunotolerance (see Roussev and Coulam, J Assist Reprod Genet. 2007 July; 24(7): 288-295). Moreover, it is known that a preimplanation embryo expresses soluble HLA-G and soluble HLA-F. The higher the expression levels of soluble HLA-G and soluble HLA-F the higher the implantation rate of the embryo. It is therefore expected that also high levels of HLA-H expression coincide with successful implantation whereas low levels of HLA-H expression coincide with implantation failure.

The present invention relates in a second aspect to an inhibitor of the nucleic acid molecule as defined in connection with the first aspect of the invention and/or a binding molecule of the protein as defined in connection with the first aspect of the invention, preferably an inhibitor of the protein as defined in connection with the first aspect of the invention for use as an immunoactivator, preferably for use in the treatment of a tumor.

A binding molecule of the protein in accordance with the invention is a compound being capable of binding to the protein in accordance with the invention. The binding molecule preferably specifically binds to the protein in accordance with the invention. Specific binding designates that the binding molecule essentially does not or essentially does not bind to other proteins or peptides than the protein in accordance with the invention. In particular, it is preferred that the binding molecule is not capable to bind to other HLA proteins than HLA-H. A binding molecule of the protein in accordance with the invention is, for example, suitable for research purposes. For example, an antibody binding to the protein in accordance with the invention can be used in immunoassays, such as an ELISA or Western Blot. The binding molecule of the protein in accordance with the invention is preferably capable of inhibiting the protein in accordance with the invention. In this case the binding molecule is designated inhibitor.

A compound inhibiting the expression of the nucleic acid molecule and/or the protein in accordance with the invention is in accordance with the present invention (i) a compound lowering or preventing the transcription of the gene encoding the nucleic acid molecule and/or the protein in accordance with invention, or (ii) is a compound lowering or preventing the translation of the mRNA encoding the protein in accordance with invention. Compounds of (i) include compounds interfering with the transcriptional machinery and/or its interaction with the promoter of said gene and/or with expression control elements remote from the promoter such as enhancers. Compounds of (ii) include compounds interfering with the translational machinery. The compound inhibiting the expression of the nucleic acid molecule and/or the protein in accordance with the invention specifically inhibits the expression of the nucleic acid molecule and/or the protein in accordance with invention, for example, by specifically interfering with the promoter region controlling the expression. Preferably, the transcription of the nucleic acid molecule and/or the protein in accordance with the invention or the translation in accordance with protein in accordance with the invention is reduced by at least 50%, more preferred at least 75% such as at least 90% or 95%, even more preferred at least 98% and most preferred by about 100% (e.g., as compared to the same experimental set up in the absence of the compound).

A compound inhibiting the activity of the nucleic acid molecule and/or the protein in accordance with the present invention causes said nucleic acid molecule and/or protein to perform its/their function with lowered efficiency. The compound inhibiting the activity of the nucleic acid molecule and/or the protein in accordance with the invention specifically inhibits the activity of said nucleic acid molecule and/or protein. As will be further detailed herein below, the compound inhibiting the activity of the nucleic acid molecule and/or the protein in accordance with the invention may specifically inhibit the activity of said nucleic acid molecule and/or protein by interacting with the nucleic acid molecule and/or protein itself or by specifically inhibiting (preferably killing) cells that produce said nucleic acid molecule and/or produce said protein and/or bind to said protein. Preferably, the activity of the nucleic acid molecule and/or the protein in accordance with the invention is reduced by at least 50%, more preferred at least 75% such as at least 90% or 95%, even more preferred at least 98%, and most preferably about 100% (e.g., as compared to the same experimental set up in the absence of the compound).

The activity of the nucleic acid molecule and/or the protein in accordance with the invention is in accordance with this invention, preferably its/their capability to induce resistance to chemotherapy in cancer patients and/or to reduce progression free as well as overall survival in cancer patients (see also the appended examples). The chemotherapy as referred to herein may be an adjuvant chemotherapy or a neoadjuvant chemotherapy, and is preferably a neoadjuvant chemotherapy. Chemotherapy uses drugs to destroy cancer cells, stop their growth, or ameliorate symptoms. In neoadjuvant (also called preoperative or primary) chemotherapy, drug treatment takes place before surgical extraction of a tumor. This is in contrast with adjuvant chemotherapy, which is drug treatment after surgery. Means and methods for determining this activity are established in the art and are illustrated in the examples herein below. In accordance with the medical aspects of the invention, these activities of the nucleic acid molecule and/or the protein in accordance with the invention are therefore to be inhibited.

The efficiency of inhibition of an inhibitor can be quantified by methods comparing the level of activity in the presence of the inhibitor to that in the absence of the inhibitor. For example, the change in the amount of the nucleic acid molecule and/or the protein in accordance with the invention formed may be used in the measurement. The efficiency of several inhibitors may be determined simultaneously in high-throughput formats. High-throughput assays, independently of being biochemical, cellular or other assays, generally may be performed in wells of microtiter plates, wherein each plate may contain 96, 384 or 1536 wells. Handling of the plates, including incubation at temperatures other than ambient temperature, and bringing into contact of test compounds with the assay mixture is preferably effected by one or more computer-controlled robotic systems including pipetting devices. In case large libraries of test compounds are to be screened and/or screening is to be effected within a short time, mixtures of, for example 10, 20, 30, 40, 50 or 100 test compounds may be added to each well. In case a well exhibits the expected activity, said mixture of test compounds may be de-convoluted to identify the one or more test compounds in said mixture giving rise to said activity.

The compounds inhibiting the expression and/or the activity of the nucleic acid molecule and/or the protein in accordance with the invention may be formulated as vesicles, such as liposomes or exososmes. Liposomes have attracted great interest because of their specificity and the duration of action they offer from the standpoint of drug delivery. Liposomal cell-type delivery systems have been used to effectively deliver nucleic acids, such as siRNA in vivo into cells (Zimmermann et al. (2006) Nature, 441:111-114). Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are phagocytosed by macrophages and other cells in vivo. Exosomes are lipid packages which can carry a variety of different molecules including RNA (Alexander et al. (2015), Nat Commun; 6:7321). The exosomes including the molecules comprised therein can be taken up by recipient cells. Hence, exosomes are important mediators of intercellular communication and regulators of the cellular niche. Exosomes are useful for diagnostic and therapeutic purposes, since they can be used as delivery vehicles, e.g. for contrast agents or drugs.

The compounds inhibiting the expression and/or the activity of the nucleic acid molecule and/or the protein in accordance with the invention can be administered to the subject at a suitable dose and/or a therapeutically effective amount. This will be further discussed herein below in connection with the pharmaceutical composition of the invention.

The length of treatment needed to observe changes and the interval following treatment for responses to occur vary depending on the desired effect. The particular amounts may be determined by conventional tests which are well known to the person skilled in the art. Suitable tests are, for example, described in Tamhane and Logan (2002), “Multiple Test Procedures for Identifying the Minimum Effective and Maximum Safe Doses of a Drug”, Journal of the American statistical association, 97(457):1-9.

The compounds inhibiting the expression and/or the activity of the nucleic acid molecule and/or the protein in accordance with the invention are preferably admixed with a pharmaceutically acceptable carrier or excipient to form a pharmaceutical composition. Suitable pharmaceutically acceptable carriers or excipients as well as the formulation of pharmaceutical compositions have been discussed herein above.

An immunoactivator is a drug being capable of promoting the immune response. Immunoactivators can be used in immunoactivating therapy, for example, to promote and/or initiate an immune response against diseased cells. The immune response is preferably a cytotoxic immune response and/or a T-cell response against the diseased cells.

As mentioned, the immunoactivator is preferably used in the context of the treatment of a tumor. As is evident from the appended examples, HLA-H is expressed in tumors. HLA-H is a secreted protein and the data in the examples herein below show that HLA-H is secreted by tumor cells, whereby most likely a “cloud” of HLA-H proteins is formed around the tumor cells, which cloud protects the tumor cells from being recognized and removed by the immune system. The binding molecule, preferably the inhibitor of the invention takes away this protective cloud from the tumor cells, thereby promoting and/or initiating an immune response against tumor cells. This immunoactivating mechanism applies mutatis mutandis to other diseased cells than tumor cells.

A tumor is an abnormal benign or malignant new growth of tissue that possesses no physiological function and arises from uncontrolled usually rapid cellular proliferation. A solid tumor is an abnormal mass of tissue that usually does not contain cysts or liquid areas by contrast to a non-solid (or liquid) tumor.

As discussed herein above, based on the data in the examples herein below it can be safely assumed that HLA-H expression is used by tumors for escaping the immune system and for becoming resistant to established anti-tumor therapies, such as chemotherapy and immune checkpoint therapy. HLA-H is believed to help the tumors by acting as an immunosuppressant. It therefore can also be safely assumed that an inhibitor of HLA-H is suitable to be used as an immunoactivator, in particular for treatment of tumors.

It is preferred that the inhibitor of HLA-H is used in combination with an established anti-tumor therapy, preferably chemotherapy or an immune checkpoint therapy, more preferably an immune checkpoint therapy and most preferably an anti-PD-L1 therapy. In Example 1 a positive correlation of HLA-H expression and the immune checkpoint PD-L1 is shown and in Example 2 it is further shown that tumor patients expressing high levels of HLA-H have a reduced survival rate when treated with an anti-PD-L1 antibody. This shows that patients expressing both, PD-L1 and HLA-H have to be treated by an anti-PD-L1 therapy as well as an HLA-H inhibitor in order to prevent the anti-PD-L1 therapy from failing.

In accordance with a preferred embodiment of the second aspect of the invention (I) the inhibitor of the nucleic acid molecule is selected from a small molecule, an aptamer, a siRNA, a shRNA, a miRNA, a ribozyme, an antisense nucleic acid molecule, a CRISPR-Cas9-based construct, a CRISPR-Cpf1-based construct, a meganuclease, a zinc finger nuclease, and a transcription activator-like (TAL) effector (TALE) nuclease, and/or (II) the binding molecule of the protein, preferably the inhibitor of the protein is selected from a small molecule, an antibody or antibody mimetic, an aptamer, wherein the antibody mimetic is preferably selected from affibodies, adnectins, anticalins, DARPins, avimers, nanofitins, affilins, Kunitz domain peptides, Fynomers®, trispecific binding molecules and probodies.

The “small molecule” as used herein is preferably an organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. The organic molecule is preferably an aromatic molecule and more preferably a heteroaromatic molecule. In organic chemistry, the term aromaticity is used to describe a cyclic (ring-shaped), planar (flat) molecule with a ring of resonance bonds that exhibits more stability than other geometric or connective arrangements with the same set of atoms. Aromatic molecules are very stable, and do not break apart easily to react with other substances. In a heteroaromatic molecule at least one of the atoms in the aromatic ring is an atom other than carbon, e.g. N, S, or O. For all above-described organic molecules the molecular weight is preferably in the range of 200 Da to 1500 Da and more preferably in the range of 300 Da to 1000 Da.

Alternatively, the “small molecule” in accordance with the present invention may be an inorganic compound. Inorganic compounds are derived from mineral sources and include all compounds without carbon atoms (except carbon dioxide, carbon monoxide and carbonates). Preferably, the small molecule has a molecular weight of less than about 2000 Da, or less than about 1000 Da such as less than about 500 Da, and even more preferably less than about Da amu. The size of a small molecule can be determined by methods well-known in the art, e.g., mass spectrometry. The small molecules may be designed, for example, based on the crystal structure of the target molecule, where sites presumably responsible for the biological activity can be identified and verified in in vivo assays such as in vivo high-throughput screening (HTS) assays.

The term “antibody” as used in accordance with the present invention comprises, for example, polyclonal or monoclonal antibodies. Furthermore, also derivatives or fragments thereof, which still retain the binding specificity to the target, e.g. the HLA-H protein of SEQ ID NO: 1 or 54, are comprised in the term “antibody”. Antibody fragments or derivatives comprise, inter alia, Fab or Fab′ fragments, Fd, F(ab′)2, Fv or scFv fragments, single domain VH or V-like domains, such as VhH or V-NAR-domains, as well as multimeric formats such as minibodies, diabodies, tribodies or triplebodies, tetrabodies or chemically conjugated Fab′-multimers (see, for example, Harlow and Lane “Antibodies, A Laboratory Manual”, Cold Spring Harbor Laboratory Press, 198; Harlow and Lane “Using Antibodies: A Laboratory Manual” Cold Spring Harbor Laboratory Press, 1999; Altshuler E P, Serebryanaya D V, Katrukha A G. 2010, Biochemistry (Mosc)., vol. 75(13), 1584; Holliger P, Hudson P J. 2005, Nat Biotechnol., vol. 23(9), 1126). The multimeric formats in particular comprise bispecific antibodies that can simultaneously bind to two different types of antigen. The first antigen can be found on the protein in accordance with the invention. The second antigen may, for example, be a tumor marker that is specifically expressed on cancer cells or a certain type of cancer cells. Non-limiting examples of bispecific antibodies formats are Biclonics (bispecific, full length human IgG antibodies), DART (Dual-affinity Re-targeting Antibody) and BiTE (consisting of two single-chain variable fragments (scFvs) of different antibodies) molecules (Kontermann and Brinkmann (2015), Drug Discovery Today, 20(7):838-847).

The term “antibody” also includes embodiments such as chimeric (human constant domain, non-human variable domain), single chain and humanised (human antibody with the exception of non-human CDRs) antibodies.

Various techniques for the production of antibodies are well known in the art and described, e.g. in Harlow and Lane (1988) and (1999) and Altshuler et al., 2010, loc. cit. Thus, polyclonal antibodies can be obtained from the blood of an animal following immunisation with an antigen in mixture with additives and adjuvants and monoclonal antibodies can be produced by any technique which provides antibodies produced by continuous cell line cultures. Examples for such techniques are described, e.g. in Harlow E and Lane D, Cold Spring Harbor Laboratory Press, 1988; Harlow E and Lane D, Using Antibodies: A Laboratory Manual, Cold Spring Harbor Laboratory Press, 1999 and include the hybridoma technique originally described by Köhler and Milstein, 1975, the trioma technique, the human B-cell hybridoma technique (see e.g. Kozbor D, 1983, Immunology Today, vol. 4, 7; Li J, et al. 2006, PNAS, vol. 103(10), 3557) and the EBV-hybridoma technique to produce human monoclonal antibodies (Cole et al., 1985, Alan R. Liss, Inc, 77-96). Furthermore, recombinant antibodies may be obtained from monoclonal antibodies or can be prepared de novo using various display methods such as phage, ribosomal, mRNA, or cell display. A suitable system for the expression of the recombinant (humanised) antibodies may be selected from, for example, bacteria, yeast, insects, mammalian cell lines or transgenic animals or plants (see, e.g., U.S. Pat. No. 6,080,560; Holliger P, Hudson P J. 2005, Nat Biotechnol., vol. 23(9), 11265). Further, techniques described for the production of single chain antibodies (see, inter alia, U.S. Pat. No. 4,946,778) can be adapted to produce single chain antibodies specific for an epitope of HLA-H. Surface plasmon resonance as employed in the BIAcore system can be used to increase the efficiency of phage antibodies.

As used herein, the term “antibody mimetics” refers to compounds which, like antibodies, can specifically bind antigens, such the HLA-H protein of SEQ ID NO: 1 or 54 in the present case, but which are not structurally related to antibodies. Antibody mimetics are usually artificial peptides or proteins with a molar mass of about 3 to 20 kDa. For example, an antibody mimetic may be selected from the group consisting of affibodies, adnectins, anticalins, DARPins, avimers, nanofitins, affilins, Kunitz domain peptides, Fynomers®, trispecific binding molecules and prododies. These polypeptides are well known in the art and are described in further detail herein below.

The term “affibody”, as used herein, refers to a family of antibody mimetics which is derived from the Z-domain of staphylococcal protein A. Structurally, affibody molecules are based on a three-helix bundle domain which can also be incorporated into fusion proteins. In itself, an affibody has a molecular mass of around 6 kDa and is stable at high temperatures and under acidic or alkaline conditions. Target specificity is obtained by randomisation of 13 amino acids located in two alpha-helices involved in the binding activity of the parent protein domain (Feldwisch J, Tolmachev V.; (2012) Methods Mol Biol. 899:103-26).

The term “adnectin” (also referred to as “monobody”), as used herein, relates to a molecule based on the 10th extracellular domain of human fibronectin III (10Fn3), which adopts an Ig-like β-sandwich fold of 94 residues with 2 to 3 exposed loops, but lacks the central disulphide bridge (Gebauer and Skerra (2009) Curr Opinion in Chemical Biology 13:245-255). Adnectins with the desired target specificity, i.e. against HLA-H, can be genetically engineered by introducing modifications in specific loops of the protein.

The term “anticalin”, as used herein, refers to an engineered protein derived from a lipocalin (Beste G, Schmidt F S, Stibora T, Skerra A. (1999) Proc Natl Acad Sci USA. 96(5):1898-903; Gebauer and Skerra (2009) Curr Opinion in Chemical Biology 13:245-255). Anticalins possess an eight-stranded β-barrel which forms a highly conserved core unit among the lipocalins and naturally forms binding sites for ligands by means of four structurally variable loops at the open end. Anticalins, although not homologous to the IgG superfamily, show features that so far have been considered typical for the binding sites of antibodies: (i) high structural plasticity as a consequence of sequence variation and (ii) elevated conformational flexibility, allowing induced fit to targets with differing shape.

As used herein, the term “DARPin” refers to a designed ankyrin repeat domain (166 residues), which provides a rigid interface arising from typically three repeated β-turns. DARPins usually carry three repeats corresponding to an artificial consensus sequence, wherein six positions per repeat are randomised. Consequently, DARPins lack structural flexibility (Gebauer and Skerra, 2009).

The term “avimer”, as used herein, refers to a class of antibody mimetics which consist of two or more peptide sequences of 30 to 35 amino acids each, which are derived from A-domains of various membrane receptors and which are connected by linker peptides. Binding of target molecules occurs via the A-domain and domains with the desired binding specificity, i.e. for HLA-H, can be selected, for example, by phage display techniques. The binding specificity of the different A-domains contained in an avimer may, but does not have to be identical (Weidle U H, et al., (2013), Cancer Genomics Proteomics; 10(4):155-68).

A “nanofitin” (also known as affitin) is an antibody mimetic protein that is derived from the DNA binding protein Sac7d of Sulfolobus acidocaldarius. Nanofitins usually have a molecular weight of around 7 kDa and are designed to specifically bind a target molecule, such as e.g. HLA-H, by randomising the amino acids on the binding surface (Mouratou B, Behar G, Paillard-Laurance L, Colinet S, Pecorari F., (2012) Methods Mol Biol.; 805:315-31).

The term “affilin”, as used herein, refers to antibody mimetics that are developed by using either gamma-B crystalline or ubiquitin as a scaffold and modifying amino-acids on the surface of these proteins by random mutagenesis. Selection of affilins with the desired target specificity, i.e. against HLA-H, is effected, for example, by phage display or ribosome display techniques. Depending on the scaffold, affilins have a molecular weight of approximately 10 or 20 kDa. As used herein, the term affilin also refers to di- or multimerised forms of affilins (Weidle U H, et al., (2013), Cancer Genomics Proteomics; 10(4):155-68).

A “Kunitz domain peptide” is derived from the Kunitz domain of a Kunitz-type protease inhibitor such as bovine pancreatic trypsin inhibitor (BPTI), amyloid precursor protein (APP) or tissue factor pathway inhibitor (TFPI). Kunitz domains have a molecular weight of approximately 6 kDA and domains with the required target specificity, i.e. against HLA-H, can be selected by display techniques such as phage display (Weidle et al., (2013), Cancer Genomics Proteomics; 10(4):155-68).

As used herein, the term “Fynomer®” refers to a non-immunoglobulin-derived binding polypeptide derived from the human Fyn SH3 domain. Fyn SH3-derived polypeptides are well-known in the art and have been described e.g. in Grabulovski et al. (2007) JBC, 282, p. 3196-3204, WO 2008/022759, Bertschinger et al (2007) Protein Eng Des Sel 20(2):57-68, Gebauer and Skerra (2009) Curr Opinion in Chemical Biology 13:245-255, or Schlatter et al. (2012), MAbs 4:4, 1-12).

The term “trispecific binding molecule” as used herein refers to a polypeptide molecule that possesses three binding domains and is thus capable of binding, preferably specifically binding to three different epitopes. At least one of these three epitopes is an epitope of the protein in accordance with the present invention. The two other epitopes may also be epitopes of the protein in accordance with the present invention or may be epitopes of one or two different antigens. The trispecific binding molecule is preferably a TriTac. A TriTac is a T-cell engager for solid tumors which comprised of three binding domains being designed to have an extended serum half-life and be about one-third the size of a monoclonal antibody.

As used herein, the term “probody” refers to a protease-activatable antibody prodrug. A probody consists of an authentic IgG heavy chain and a modified light chain. A masking peptide is fused to the light chain through a peptide linker that is cleavable by tumor-specific proteases. The masking peptide prevents the probody binding to healthy tissues, thereby minimizing toxic side effects.

Aptamers are nucleic acid molecules or peptide molecules that bind a specific target molecule. Aptamers are usually created by selecting them from a large random sequence pool, but natural aptamers also exist in riboswitches. Aptamers can be used for both basic research and clinical purposes as macromolecular drugs. Aptamers can be combined with ribozymes to self-cleave in the presence of their target molecule. These compound molecules have additional research, industrial and clinical applications (Osborne et. al. (1997), Current Opinion in Chemical Biology, 1:5-9; Stull & Szoka (1995), Pharmaceutical Research, 12, 4:465-483).

Nucleic acid aptamers are nucleic acid species that normally consist of (usually short) strands of oligonucleotides. Typically, they have been engineered through repeated rounds of in vitro selection or equivalently, SELEX (systematic evolution of ligands by exponential enrichment) to bind to various molecular targets such as small molecules, proteins, nucleic acids, and even cells, tissues and organisms.

Peptide aptamers are usually peptides or proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). The variable peptide loop typically comprises 10 to 20 amino acids, and the scaffold may be any protein having good solubility properties. Currently, the bacterial protein Thioredoxin-A is the most commonly used scaffold protein, the variable peptide loop being inserted within the redox-active site, which is a -Cys-Gly-Pro-Cys-loop (SEQ ID NO: 8) in the wild protein, the two cysteins lateral chains being able to form a disulfide bridge. Peptide aptamer selection can be made using different systems, but the most widely used is currently the yeast two-hybrid system.

Aptamers offer the utility for biotechnological and therapeutic applications as they offer molecular recognition properties that rival those of the commonly used biomolecules, in particular antibodies. In addition to their discriminatory recognition, aptamers offer advantages over antibodies as they can be engineered completely in a test tube, are readily produced by chemical synthesis, possess desirable storage properties, and elicit little or no immunogenicity in therapeutic applications. Non-modified aptamers are cleared rapidly from the bloodstream, with a half-life of minutes to hours, mainly due to nuclease degradation and clearance from the body by the kidneys, a result of the aptamers' inherently low molecular weight. Unmodified aptamer applications currently focus on treating transient conditions such as blood clotting, or treating organs such as the eye where local delivery is possible. This rapid clearance can be an advantage in applications such as in vivo diagnostic imaging. Several modifications, such as 2′-fluorine-substituted pyrimidines, polyethylene glycol (PEG) linkage, fusion to albumin or other half life extending proteins etc. are available to scientists such that the half-life of aptamers can be increased for several days or even weeks.

As discussed, the above-described small molecule, antibody or antibody mimetic and aptamer can specifically bind to the protein in accordance with the present invention. This binding may block the immunosuppressive properties of the protein in accordance with the present invention and preferably its capability to induce resistance to chemotherapy in cancer patients and/or to reduce progression free as well as overall survival in cancer patients. In this case the small molecule, antibody or antibody mimetic and aptamer are also referred to as blocking small molecule, antibody or antibody mimetic and aptamer. A blocking small molecule, antibody or antibody mimetic and aptamer blocks interactions of the protein in accordance with the present invention with other cellular components, such as ligands and receptor which normally interact with the protein in accordance with the present invention.

The small molecule, antibody or antibody mimetic and aptamer can also be generated in the format of drug-conjugates. In this case the small molecule, antibody or antibody mimetic and aptamer in itself may not have an inhibitory effect but the inhibitory effect is only conferred by the drug. The small molecule, antibody or antibody mimetic and aptamer confer the site-specificity binding of the drug to cells producing and/or binding to the protein in accordance with the present invention. The drug is preferably capable to kill cells producing and/or binding to the protein in accordance with the invention. Hence, by combining the targeting capabilities of molecules binding to the protein in accordance with the present invention with the cell-killing ability of the drug, the drug conjugates become inhibitors that allow for discrimination between healthy and diseased tissue and cells. Cleavable and non-cleavable linkers to design drug conjugates are known in the art. Non-limiting examples of drugs being capable of killing cells are cytostatic drugs and radioisotopes that deliver radiation directly to the cancer cells.

It is furthermore possible to confine the binding and/or inhibitory activity of the small molecule, antibody or antibody mimetic and aptamer to certain tissues or cell-types, in particular diseased tissues or cell-types. For instance, probodies may be designed. In a probody the small molecule, antibody or antibody mimetic or aptamer is bound to a masking peptide which limits or prevents binding to the protein in accordance with the invention and which masking peptide can be cleaved by a protease. Proteases are enzymes that digest proteins into smaller pieces by cleaving specific amino acid sequences known as substrates. In normal healthy tissue, protease activity is tightly controlled. In cancer cells, protease activity is upregulated. In healthy tissue or cells, where protease activity is regulated and minimal, the target-binding region of the probody remains masked and is thus unable to bind. On the other hand, in diseased tissue or cells, where protease activity is upregulated, the target-binding region of the probody gets unmasked and is thus able to bind and/or inhibit.

In accordance with the present invention, the term “small interfering RNA (siRNA)”, also known as short interfering RNA or silencing RNA, refers to a class of 18 to 30, preferably 19 to 25, most preferred 21 to 23 or even more preferably 21 nucleotide-long double-stranded RNA molecules that play a variety of roles in biology. Most notably, siRNA is involved in the RNA interference (RNAi) pathway where the siRNA interferes with the expression of a specific gene. In addition to their role in the RNAi pathway, siRNAs also act in RNAi-related pathways, e.g. as an antiviral mechanism or in shaping the chromatin structure of a genome.

siRNAs naturally found in nature have a well defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest. Essentially any gene for which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, it is preferred that at least one RNA strand has a 5′- and/or 3′-overhang. Preferably, one end of the double-strand has a 3′-overhang from 1 to 5 nucleotides, more preferably from 1 to 3 nucleotides and most preferably 2 nucleotides. The other end may be blunt-ended or has up to 6 nucleotides 3′-overhang. In general, any RNA molecule suitable to act as siRNA is envisioned in the present invention. The most efficient silencing was so far obtained with siRNA duplexes composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair (Elbashir et al. 2001). 2′-deoxynucleotides in the 3′ overhangs are as efficient as ribonucleotides, but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as for example 5% glucose) or cationic lipids and polymers can be used for siRNA delivery in vivo through systemic routes either intravenously (IV) or intraperitoneally (IP) (De Fougerolles et al. (2008), Current Opinion in Pharmacology, 8:280-285; Lu et al. (2008), Methods in Molecular Biology, vol. 437: Drug Delivery Systems—Chapter 3: Delivering Small Interfering RNA for Novel Therapeutics).

A short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it. si/shRNAs to be used in the present invention are preferably chemically synthesized using appropriately protected ribonucleoside phosphoramidites and a conventional DNA/RNA synthesizer. Suppliers of RNA synthesis reagents are Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). Most conveniently, siRNAs or shRNAs are obtained from commercial RNA oligo synthesis suppliers, which sell RNA-synthesis products of different quality and costs. In general, the RNAs applicable in the present invention are conventionally synthesized and are readily provided in a quality suitable for RNAi.

Further molecules effecting RNAi include, for example, microRNAs (miRNA). Said RNA species are single-stranded RNA molecules. Endogenously present miRNA molecules regulate gene expression by binding to a complementary mRNA transcript and triggering of the degradation of said mRNA transcript through a process similar to RNA interference. Accordingly, exogenous miRNA may be employed as an inhibitor of HLA-H after introduction into the respective cells.

A ribozyme (from ribonucleic acid enzyme, also called RNA enzyme or catalytic RNA) is an RNA molecule that catalyses a chemical reaction. Many natural ribozymes catalyse either their own cleavage or the cleavage of other RNAs, but they have also been found to catalyse the aminotransferase activity of the ribosome. Non-limiting examples of well-characterised small self-cleaving RNAs are the hammerhead, hairpin, hepatitis delta virus, and in vitro-selected lead-dependent ribozymes, whereas the group I intron is an example for larger ribozymes. The principle of catalytic self-cleavage has become well established in recent years. The hammerhead ribozymes are characterised best among the RNA molecules with ribozyme activity. Since it was shown that hammerhead structures can be integrated into heterologous RNA sequences and that ribozyme activity can thereby be transferred to these molecules, it appears that catalytic antisense sequences for almost any target sequence can be created, provided the target sequence contains a potential matching cleavage site. The basic principle of constructing hammerhead ribozymes is as follows: A region of interest of the RNA, which contains the GUC (or CUC) triplet, is selected. Two oligonucleotide strands, each usually with 6 to 8 nucleotides, are taken and the catalytic hammerhead sequence is inserted between them. The best results are usually obtained with short ribozymes and target sequences.

A recent development, also useful in accordance with the present invention, is the combination of an aptamer, recognizing a small compound, with a hammerhead ribozyme. The conformational change induced in the aptamer upon binding the target molecule can regulate the catalytic function of the ribozyme.

The term “antisense nucleic acid molecule”, as used herein, refers to a nucleic acid which is complementary to a target nucleic acid. An antisense molecule in accordance with the invention is capable of interacting with the target nucleic acid, more specifically it is capable of hybridizing with the target nucleic acid. Due to the formation of the hybrid, transcription of the target gene(s) and/or translation of the target mRNA is reduced or blocked. Standard methods relating to antisense technology have been described (see, e.g., Melani et al., Cancer Res. (1991) 51:2897-2901).

CRISPR/Cas9, as well as CRISPR-Cpf1, technologies are applicable in nearly all cells/model organisms and can be used for knock out mutations, chromosomal deletions, editing of DNA sequences and regulation of gene expression. The regulation of the gene expression can be manipulated by the use of a catalytically dead Cas9 enzyme (dCas9) that is conjugated with a transcriptional repressor to repress transcription a specific gene, here the HLA-H gene. Similarly, catalytically inactive, “dead” Cpf1 nuclease (CRISPR from Prevotella and Francisella-1) can be fused to synthetic transcriptional repressors or activators to downregulate endogenous promoters, e.g. the promoter which controls HLA-H expression. Alternatively, the DNA-binding domain of zinc finger nucleases (ZFNs) or transcription activator-like effector nucleases (TALENs) can be designed to specifically recognize the HLA-H gene or its promoter region or its 5-UTR thereby inhibiting the expression of the HLA-H gene.

Inhibitors provided as inhibiting nucleic acid molecules that target the HLA-H gene or a regulatory molecule involved in HLA-H expression are also envisaged herein. Such molecules, which reduce or abolish the expression of HLA-H or a regulatory molecule include, without being limiting, meganucleases, zinc finger nucleases and transcription activator-like (TAL) effector (TALE) nucleases. Such methods are described in Silva et al., Curr Gene Ther. 2011; 11(1):11-27; Miller et al., Nature biotechnology. 2011; 29(2):143-148, and Klug, Annual review of biochemistry. 2010; 79:213-231.

In connection with the second aspect of the invention, the binding molecule of the protein as defined in connection with the first aspect, preferably the inhibitor of the protein as defined in connection with the first aspect may also be a cell such as a T-cell, wherein the T-cell is preferably a CAR-T-cell.

The cell generally carries on its surface a binding molecule, preferably an inhibitor of the protein as defined in connection with the first aspect. In the case of a T-cell the binding molecule, preferably the inhibitor is a naturally occurring or chimeric T-cell receptor that specifically targets the protein as defined in connection with the first aspect. Chimeric antigen receptor T-cells (also known as CAR T-cells) are T-cells that have been genetically engineered to produce an artificial T-cell receptor for use in immunotherapy.

Chimeric antigen receptors (CARs, also known as chimeric immunoreceptors, chimeric T cell receptors or artificial T cell receptors) are accordingly receptor proteins that have been engineered to give T-cells the new ability to specifically target the protein as defined in connection with the first aspect. The receptors are chimeric because they combine both antigen-binding and T-cell activating functions into a single receptor.

The present invention relates in a third aspect to the use of the nucleic acid molecule as defined in item (I)(g) of the first aspect of the invention or the protein or peptide as defined in connection with the first aspect of the invention in a sample obtained from a subject for diagnosing a tumor and/or for grading a tumor and/or for tumor prognosis and/or classifying tumor as a HLA-H low expression tumor or a HLA-H high expression tumor and/or for diagnosing an implantation failure.

The sample may be a body fluid of the subject or a tissue sample from an organ of the subject. Non-limiting examples of body fluids are whole blood, blood plasma, blood serum, urine, peritoneal fluid, and pleural fluid, liquor cerebrospinalis, tear fluid, or cells therefrom in solution. Non-limiting examples of tissue are colon, liver, breast, ovary, and testis. Tissue samples may be taken by aspiration or punctuation, excision or by any other surgical method leading to biopsy or resected cellular material. The sample may be a processed sample, e.g. a sample which has been frozen, fixed, embedded or the like. A preferred type of sample is a formaline fixed paraffin embedded (FFPE) sample. Preparation of FFPE samples are standard medical practice and these samples can be conserved for long periods of time.

The term “diagnosing” as used herein is directed to the identification of a disease in a subject suffering from symptoms of a disease. In accordance with the invention the disease is a tumor or an implantation failure. The term “grading” as used herein is directed to the identification of the degree of cell anaplasia of a tumor cell in a subject which has been diagnosed to have a tumor. The most commonly system used for grading tumors is the system according to the guidelines of the American Joint Commission on Cancer. As per these guidelines, the following grading categories are distinguished: GX (grade cannot be assessed), G1 (well-differentiated; low grade), G2 (moderately differentiated; intermediate grade), G3 (poorly differentiated, high grade); G4 (undifferentiated, high grade). The term “prognosis” as used herein is directed to the outlook or chance of recovery from a disease such as a tumor and/or is the outlook or chance of survival of a disease, such as a tumor. In the case of a tumor, the prognosis may comprise one or more of tumor size alteration of target lesion, disease-specific survival (DSS), recurrence-free survival (RFS), progression-free survival (PFS) and distant recurrence-free survival, wherein DSS is preferred.

The term “subject” in accordance with the invention refers to a mammal, preferably a domestic animal or a pet animal such as horse, cattle, pig, sheep, goat, dog or cat, and most preferably a human.

As discussed above, increased level of HLA-H expression in tumor patients is associated with a significantly reduced progression free survival and overall survival of tumor patients. Accordingly, higher levels of HLA-H expression also coincide with higher tumor grades. It is furthermore demonstrated in the examples that HLA-H expression was found in all tumor samples, so that HLA-H expression can not only serve as prognostic marker but also as diagnostic marker for tumors.

In the above use a positive and/or a negative sample as well as predetermined standards may be incorporated. The controls may be obtained from sample of one or more subjects, such as at least 5, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 1500, or at least 2000 subjects. Predetermined standards designate previously obtained values from a positive and/or a negative sample(s).

For the diagnosis of a tumor it is believed that no standard at all is required since it is expected that healthy subjects do not express HLA-H. Moreover, it is shown in the examples that HLA-H expression was detected in all investigated tumor patients. Notwithstanding a positive and/or a negative sample as well as predetermined standards may be incorporated in the diagnostic tumor application of the present invention. For diagnosis the positive sample is from one or more subjects known to have a tumor, preferably a tumor of the same body site as the one to be diagnosed. Similarly, the negative sample is from one or more subjects known to have no tumor. A subject is diagnosed to have a tumor if the expression level of the nucleic acid molecule in accordance with the invention or the protein or peptide in accordance with the invention in a sample is with increased preference at least 1.5-fold, 2-fold, 3-fold, 4-fold increased as compared to the negative control or a predetermined standard derived therefrom. A subject is diagnosed to have a tumor if the expression level of the nucleic acid molecule in accordance with the invention or the protein or peptide in accordance with the invention in a sample is with increased preference less than 50%, less than 25% and less than 10% different from the positive control or a predetermined standard derived therefrom. For example, if the positive control is set to 100%, a patient displaying values of 150% to 50%, preferably 125% or 75% is diagnosed to have a tumor.

Also for the diagnosis of an implantation failure the positive and/or a negative sample as well as predetermined standards may be used. For diagnosis the positive sample is from one or more female subjects that had at least one implantation failure, preferably at least two implantation failures and most preferably at least three implantation failures. Two or more implantation failures are also referred as repetitive or recurrent implantation failure. Similarly, the negative sample is from one or more female subjects that had at least one successful pregnancy, preferably at least two successful pregnancy, and most preferably at least three successful pregnancies. A female subject is diagnosed to have an implantation failure if the expression level of the nucleic acid molecule of the first aspect or the protein or peptide of the fourth aspect in a sample is with increased preference at least 1.5-fold, 2-fold, 3-fold, 4-fold decreased as compared to the negative control or a predetermined standard derived therefrom. A subject is diagnosed to have an implantation failure if the expression level of the nucleic acid molecule in accordance with the invention or the protein or peptide in accordance with the invention in a sample is with increased preference less than 50%, less than 25% and less than 10% different (i.e. higher or lower) from the positive control or a predetermined standard derived therefrom. For example, if the positive control is set to 100%, a patient displaying values of 125% or 75% is diagnosed to have an implantation failure.

With respect to classifying a tumor as a HLA-H low expression tumor or a HLA-H high expression tumor it is noted that not each and every tumor is expected to express or express substantial amounts of HLA-H. Hence, in order to reveal whether in a subject having a tumor a binding molecule, preferably an inhibitor in accordance with the invention can be a treatment option, the tumor may be classified as a HLA-H low expression tumor or a HLA-H high expression, wherein only in the later case the binding molecule or inhibitor is an option. For classification, the control may be from one or more subjects known to have tumor expressing HLA-H and preferably known to have tumor that was treatable by the binding molecule, preferably the inhibitor in accordance with the invention. In case the HLA-H expression of the tumor to be classified is with increasing preference at least 2-fold, at least 3-fold, at least 3-fold, at least 4-fold and at least 5-fold decreased as compared to the control the tumor is a HLA-H low expression tumor. On the other hand, in case the HLA-H expression of the tumor to be classified is with increasing preference at least 2-fold, at least 3-fold, at least 3-fold, at least 4-fold and at least 5-fold increased as compared to the control the tumor is a HLA-H high expression tumor.

For prognosis, the positive control may be from one or more subjects that died from the tumor (preferably a tumor of the same body site as the one to be prognosed) and the negative sample may be from one or more subjects that survived the tumor for a substantial amount of time without tumor progression (preferably a tumor of the same body site as the one to be prognosed). A substantial amount designates with increased preference at least 1 year, at least 2 year, at least 3 year, at least 4 year and at least 5 years. A subject has a favorable prognosis if the expression level of the nucleic acid molecule in accordance with the invention or the protein or peptide in accordance with the invention in a sample is with increased preference at least 1.5-fold, 2-fold, 3-fold, 4-fold decreased as compared to the positive control or a predetermined standard derived therefrom. Also a subject has a favorable prognosis if the expression level of the nucleic acid molecule in accordance with the invention or the protein or peptide in accordance with the invention in a sample is with increased preference less than 50%, less than 25% and less than 10% different from the negative control or a predetermined standard derived therefrom. A subject has an unfavorable prognosis if the expression level of the nucleic acid molecule in accordance with the invention or the protein or peptide in accordance with the invention in a sample is with increased preference at least 1.5-fold, 2-fold, 3-fold, 4-fold increased as compared to the negative control or a predetermined standard derived therefrom. Also a subject has an unfavorable prognosis if the expression level of the nucleic acid molecule in accordance with the invention or the protein or peptide in accordance with the invention in a sample is with increased preference less than 50%, less than 25% and less than 10% different from the positive control or a predetermined standard derived therefrom. The prognosis is preferably the prognosis of the expected treatment success of a tumor treatment, wherein the anti-tumor treatment is preferably chemotherapy and/or the patients to be diagnosed has preferably a breast cancer.

For grading, the positive sample may be from one or more subjects that are graded to one of the categories G1 to G4. For grading more than one positive sample can be used, wherein the positive samples are from two, preferably three and most preferably all four of categories G1 to G4. A subject is graded as having a G1 tumor if the expression level of the nucleic acid molecule in accordance with the invention or the protein or peptide in accordance with the invention in a sample is with increased preference less than 50%, less than 25% and less than 10% different from the positive G1 control or a predetermined standard derived therefrom. This applies mutatis mutandis to stages G2 to G4.

Methods for obtaining the levels of the nucleic acid molecule in accordance with the invention or the protein or peptide in accordance with the invention as established in the art.

For instance, levels of the nucleic acid molecule in accordance with the invention may be obtained by real time quantitative PCR (RT-qPCR), electrophoretic techniques or a DNA Microarray (Roth (2002), Curr. Issues Mol. Biol., 4: 93-100), wherein RT-qPCR is preferred. In these methods the expression level may be normalized against the (mean) expression level of one or more reference genes in the sample. The term “reference gene”, as used herein, is meant to refer to a gene which has a relatively invariable level of expression on the RNA transcript/mRNA level in the system which is being examined, i.e. cancer. Such gene may be referred to as housekeeping gene. Non-limiting examples of reference genes are CALM2, B2M, RPL37A, GUSB, HPRT1 and GAPDH, preferably CALM2 and/or B2M. Other suitable reference genes are known to a person skilled in the art.

RT-qPCR is illustrated by the examples. RT-qPCR is carried out in a thermal cycler with the capacity to illuminate each sample with a beam of light of at least one specified wavelength and detect the fluorescence emitted by the excited fluorophore. The thermal cycler is also able to rapidly heat and chill samples, thereby taking advantage of the physicochemical properties of the nucleic acids and DNA polymerase. The two common methods for the detection of PCR products in real-time qPCR are: (1) non-specific fluorescent dyes that intercalate with any double-stranded DNA, and (2) sequence-specific DNA probes consisting of oligonucleotides that are labelled with a fluorescent reporter which permits detection only after hybridization of the probe with its complementary sequence (e.g. a TaqMan probe). The latter detection method is used in the examples herein below. The probes are generally fluorescently labeled probes. Preferably, the fluorescently labeled probe consists of an oligonucleotide labeled with both a fluorescent reporter dye and a quencher dye (=dual-label probe). Suitable fluorescent reporter and quencher dyes/moieties are known to a person skilled in the art and include, but are not limited to the reporter dyes/moieties 6-FAM™, JOE™, Cy5®, Cy3® and the quencher dyes/moieties dabcyl, TAMRA™, BHQ™-1, -2 or -3. Preferably primers for use in accordance with the present invention have a length of 15 to 30 nucleotides, and are in particular deoxyribonucleotides. In one embodiment, the primers are designed so as to (1) be specific for the target mRNA-sequence of HLA-H or being derived therefrom, (2) provide an amplicon size of less than 120 bp (preferably less than 100 bp), (3) be mRNA-specific (consideration of exons/introns; preferably no amplification of genomic DNA), (4) have no tendency to dimerize and/or (5) have a melting temperature Tₘ in the range of from 58° C. to 62° C. (preferably, Tₘ is approximately 60° C.).

As an alternative of qPCR also electrophoretic techniques or a DNA microarray may be used to obtaining the levels of the nucleic acid molecule in accordance with the invention. The conventional approach to mRNA identification and quantitation is through a combination of gel electrophoresis, which provides information on size, and sequence-specific probing. The Northern blot is the most commonly applied technique in this class. The ribonuclease protection assay (RPA) was developed as a more sensitive, less labor-intensive alternative to the Northern blot. Hybridization is performed with a labeled ribonucleotide probe in solution, after which non-hybridized sample and probe are digested with a mixture of ribonucleases (e.g., RNase A and RNase T1) that selectively degrade single-stranded RNAs. Subsequent denaturing polyacrylamide gel electrophoresis provides a means for quantitation and also gives the size of the region hybridized by the probe. For both Northern blot and RPA, the accuracy and precision of quantitation are functions of the detection method and the reference or standard utilized.

Most commonly, the probes are radiolabeled with 32P or 33P, in which case the final gel is exposed to X-ray film or phosphor screen and the intensity of each band quantified with a densitometer or phosphor imager, respectively. In both cases, the exposure time can be adjusted to suit the sensitivity required, but the phosphorbased technique is generally more sensitive and has a greater dynamic range. As an alternative to using radioactivity, probes can be labeled with an antigen or hapten, which is subsequently bound by a horseradish peroxidase- or alkaline phosphatase-conjugated antibody and quantified after addition of substrate by chemiluminesence on film or a fluorescence imager. In all of these imaging applications, subtraction of the background from a neighboring region of the gel without probe should be performed. The great advantage of the gel format is that any reference standards can be imaged simultaneously with the sample. Likewise, detection of a housekeeping gene is performed under the same conditions for all samples.

For the construction of DNA microarrays two technologies have emerged. Generally, the starting point in each case for the design of an array is a set of sequences corresponding to the genes or putative genes to be probed. In the first approach, oligonucleotide probes are synthesized chemically beginning from a glass substrate. Because of the variable efficiency of oligonucleotide hybridization to cDNA probes, multiple oligonucleotide probes are synthesized complementary to each gene of interest. Furthermore, for each fully complementary oligonucleotide on the array, an oligonucleotide with a mismatch at a single nucleotide position is constructed and used for normalization. Oligonucleotide arrays are routinely created with densities of about 10⁴-10⁶ probes/cm². The second major technology for DNA microarray construction is the robotic printing of cDNA probes directly onto a glass slide or other suitable substrate. A DNA clone is obtained for each gene of interest, purified, and amplified from a common vector by PCR using universal primers. The probes are robotically deposited in spots on the order of 50-200 μm in size. At this spacing, a density of, for example, approximately 10³ probes/cm² can be achieved.

Levels of the protein or peptide in accordance with the invention may be determined, for example, by using a “molecule binding to the protein or peptide” and preferably a “molecule specifically binding to the protein or peptide”. A molecule binding to the protein or peptide designates a molecule which under known conditions occurs predominantly bound to the protein or peptide. A “molecule binding to the protein or peptide” one of the herein above described binding molecules, preferably inhibitors of the protein or peptide in accordance with the invention may be used, such as antibodies, aptamers, etc. Levels of the protein or peptide in accordance with the invention may also be obtained by using Western Blot analysis, mass spectrometry analysis, FACS-analysis, ELISA, and immunohistochemistry. These techniques are non-limiting examples of methods which may be used to qualitatively, semi-quantitatively and/or quantitatively detect a protein or peptide.

Western blot analysis is a widely used and well-know analytical technique used to detect specific proteins or peptides in a given sample, for example, a tissue homogenate or body extract. It uses gel electrophoresis to separate native or denatured proteins or peptides by the length of the (poly)peptide (denaturing conditions) or by the 3-D structure of the protein (native/non-denaturing conditions). The proteins or peptides are then transferred to a membrane (typically nitrocellulose or PVDF), where they are probed (detected) using antibodies specific to the target protein.

Also mass spectrometry (MS) analysis is a widely used and well-know analytical technique, wherein the mass-to-charge ratio of charged particles is measured. Mass spectrometry is used for determining masses of particles, for determining the elemental composition of a sample or molecule, and for elucidating the chemical structures of molecules, such as proteins, peptides and other chemical compounds. The MS principle consists of ionizing chemical compounds to generate charged molecules or molecule fragments and measuring their mass-to-charge ratios.

Fluorescence activated cell sorting (FACS) analysis is a widely used and well-known analytical technique, wherein biological cells are sorted based upon the specific light scattering of the fluorescent characteristics of each cell. Cells may be fixed in 4% formaldehyde, permeabilized with 0.2% Triton-X-100, and incubated with a fluorophore-labeled antibody (e.g. mono- or polyclonal anti-HLA-H antibody).

Enzyme-linked immunosorbent assay (ELISA) is a widely used and well-know sensitive analytical technique, wherein an enzyme is linked to an antibody or antigen as a marker for the detection of a specific protein or peptide.

Immunohistochemistry (IHC) is the most common application of immunostaining. It involves the process of selectively identifying antigens (proteins) in cells of a tissue section by exploiting the principle of antibodies binding specifically to antigens in biological tissues. In combination with particular devices IHC can be used for quantitative in situ assessment of protein expression (for review Cregger et al. (2006) Arch Pathol Lab Med, 130:1026-1030). Quantitative IHC takes advantage of the fact that staining intensity correlates with absolute protein levels.

Next to the nucleic acid molecule in accordance with the invention or the protein or peptide in accordance with the invention one or more further compounds in the sample obtained from a subject may be used for diagnosing a tumor and/or for grading a tumor and/or for tumor prognosis. A vast number of markers for diagnosing a tumor and/or for grading a tumor and/or for tumor prognosis are known in the art may be used in conjunction with the nucleic acid molecule in accordance with the invention or the protein or peptide in accordance with the invention. Several tumor markers are indicative for particular tumor, such as breast cancer or colon cancer. Tumor markes are, for example listed at the National Cancer Institute (https://www.cancer.gov/about-cancer/diagnosis-staging/diagnosis/tumor-markers-fact-sheet) or the integrated database of cancer genes and markers CGMD (http://cgmd.in/). The use of one or more further markers generally increases the reliability of the diagnosis, the grading or the prognosis.

The present invention relates in a fourth aspect to a method for diagnosing a tumor comprising detecting the presence of the nucleic acid molecule as defined in item (I)(g) of the first aspect of the invention and/or the protein or peptide as defined in connection with the first aspect of the invention in a sample obtained from a subject, wherein the presence of the nucleic acid molecule as defined in item 1(I)(g) of the first aspect of the invention and/or the protein as defined in connection with the first aspect of the invention is indicative for a tumor in the subject.

The present invention relates in a fifth aspect to a method for grading a tumor and/or for tumor prognosis comprising determining the level of the nucleic acid molecule of as defined in item (I)(g) of the first aspect of the invention and/or the protein or peptide as defined in connection with the first aspect of the invention in a sample obtained from a subject, wherein increased levels of the nucleic acid molecule as defined in item 1(I)(g) of the first aspect of the invention and/or the protein or peptide as defined in connection with the first aspect of the invention as compared to a control correlate with the a higher grade of the tumor and/or an adverse tumor prognosis.

The methods of the fourth and fifth aspect of the invention implement the use of the third aspect of the invention in the format of methods. It follows that the definitions and preferred embodiments provided herein above in connection with the third aspect of the invention are equally applicable to the fourth and fifth aspect of the invention.

The present invention relates in a sixth aspect to a kit for diagnosing a tumor and/or for grading a tumor and/or for tumor prognosis, comprising (a) means for the detection and/or quantification of the nucleic acid molecule as defined in item (I)(g) of the first aspect of the invention and/or the protein or peptide as defined in connection with the first aspect of the invention in a sample obtained from a subject, and (b) instructions for using the kit.

The kit of the sixth aspect of the invention implements a/the means required for conducting the use of the third aspect of the invention in the format of a kit. For this reason the definitions and preferred embodiments provided herein above in connection with the third aspect of the invention are equally applicable to the kit of the sixth aspect of the invention.

A/the means for the detection and/or quantification of the nucleic acid molecule of the first aspect are preferably one or more of the primer and probes for HLA_H as shown in Table 1 of the examples are used in a RT-qPCR, a specific primers pair for HLA-H which can optionally be used in connection with the respective probe. A/the means for the detection of the protein or peptide in accordance with the invention are preferably an antibody and/or antibody mimetic as described herein above. For detection and/or quantification the antibody and/or antibody mimetic may be labelled, e.g. by a fluorescent dye or a radiolabel. Examples of fluorescent dyes and radiolabels are also described herein above.

The various components of the kit may be packaged into one or more containers such as one or more vials. The vials may, in addition to the components, comprise preservatives or buffers for storage. The kit may comprise instructions how to use the kit, which preferably inform how to use the components of the kit for diagnosing a tumor and/or for grading a tumor and/or for tumor prognosis.

As also discussed herein above, the examples show that HLA-H is expressed in tumors at various stages and used by the tumors to escape the immune system. High expression levels of HLA-H are associated with inferior prognosis. In Example 4 it is furthermore shown that elevated HLA-H expression is positively associated with advanced tumor stages. Hence, detection and/or quantification of the nucleic acid molecule as defined in item (I)(g) of the first aspect of the invention and/or the protein or peptide as defined in connection with the first aspect of the invention in a sample obtained from a subject by the methods and kits as described herein above is a means for diagnosing a tumor and/or for grading a tumor and/or for tumor prognosis.

The present invention relates in a seventh aspect to a method for monitoring the non-efficacy of a tumor treatment in a subject having a tumor comprising (a) determining the amount of the nucleic acid molecule as defined in item (I)(g) of the first aspect of the invention and/or the protein or peptide as defined in connection with the first aspect of the invention in a sample obtained from a subject before the start of the treatment; and (b) determining the amount of the nucleic acid molecule as defined in item (I)(g) of the first aspect of the invention and/or the protein or peptide as defined in connection with the first aspect of the invention in a sample obtained from a subject at one or more times after the start of the treatment, wherein an increased amount in b) as compared to a) is indicative for the non-efficacy of a tumor treatment and/or a decreased amount in b) as compared to a) is indicative for the efficacy of a tumor treatment.

Similarly, the present invention relates in a ninth aspect to a method for monitoring the non-efficacy of a immunosuppressive therapy in a subject requiring such a therapy comprising (a) determining the amount of the nucleic acid molecule as defined in item (I)(g) of the first aspect of the invention and/or the protein or peptide as defined in connection with the first aspect of the invention in a sample obtained from a subject before the start of the therapy; and (b) determining the amount of the nucleic acid molecule as defined in item (I)(g) of the first aspect of the invention and/or the protein or peptide as defined in connection with the first aspect of the invention in a sample obtained from a subject at one or more times after the start of the therapy, wherein a decreased amount in b) as compared to a) is indicative for the non-efficacy of a immunosuppressive therapy and/or an increased amount in b) as compared to a) is indicative for the efficacy of a immunosuppressive therapy.

The definitions and preferred embodiments provided herein above in connection with the other aspects of the invention are equally applicable to the eighth and ninth aspect of the invention. For example, means and methods for determining the amount of the nucleic acid molecule in accordance with the invention and/or the protein or peptide in accordance with the invention have been described herein above in connection with the third aspect of the invention. These means and methods can equally be used in connection with the eighth and ninth aspect of the invention.

The tumor treatment can be any tumor treatment, for example, surgery, radiotherapy or chemotherapy. The tumor treatment is preferably chemotherapy. Chemotherapy comprises the administration of chemotherapeutic agents. Chemotherapeutic agents that can be used according to the invention include cytostatic compounds and cytotoxic compounds. Traditional chemotherapeutic agents act by killing cells that divide rapidly, one of the main properties of most tumor cells. Chemotherapeutic agents include but are not limited to taxanes, nucleoside analogs, camptothecin analogs, anthracyclines and anthracycline analogs, etoposide, bleomycin, vinorelbine, cyclophosphamide, antimetabolites, anti-mitotics, and alkylating agents. The chemotherapy may also be platinum-based, i.e. comprises the administration of platinum-based compounds, e.g., cisplatin. Chemotherapeutic agents are often given in combinations, usually for 3-6 months. One of the most common treatments comprises the administration of cyclophosphamide plus doxorubicin (adriamycin; belonging to the group of anthracyclines and anthracycline analogs), known as AC. Sometimes, a taxane drug, such as docetaxel, is added, and the regime is then known as CAT; taxane attacks the microtubules in cancer cells. Another common treatment, which produces equivalent results, comprises the administration of cyclophosphamide, methotrexate, which is an antimetabolite, and fluorouracil, which is a nucleoside analog (CMF). Another standard chemotherapeutic treatment comprises the administration of fluorouracil, epirubicin and cyclophosphamide (FEC), which may be supplemented with a taxane, such as docetaxel, or with vinorelbine.

The tumor is in accordance with the eighth aspect, preferably a non-luminal tumor. A non-luminal is a hormone-receptor (oestrogen-receptor and/or progesterone-receptor) negative tumor or a tumor expressing a low level of hormone-receptor (oestrogen-receptor and/or progesterone-receptor). In the case of breast tumors, luminal A tumors are hormone-receptor positive, Her2 negative, and express low levels of Ki-67, and luminal B tumors are (i) hormone-receptor positive, Her2 negative, and express high levels of Ki-67, or (ii) are oestrogen-receptor positive, progesterone-receptor negative, Her2 negative, and express low levels of Ki-67. The non-luminal breast tumors can be divided into HER2 positive tumors and TNBC (triple negative breast cancer), being HER2 negative and hormone-receptor (oestrogen-receptor and/or progesterone-receptor) negative.

Likewise the immunosupressive therapy can be any immunosupressive therapy. For example, the immunosupressive therapy may comprise the administration of one or more immunosuppressive drugs, e.g. selected from glucocorticoids, cytostatics and antibodies. In accordance with the fifteenth aspect, the subject may have received a transplanted organ or tissue (e.g., bone marrow, heart, kidney, liver), or may have an autoimmune diseases or a disease that is most likely of autoimmune origin (e.g., rheumatoid arthritis, multiple sclerosis, myasthenia gravis, psoriasis, vitiligo, systemic lupus erythematosus, sarcoidosis, focal segmental glomerulosclerosis, Crohn's disease, Behcet's Disease, pemphigus, ankylosing spondylitis, and ulcerative colitis) or another non-autoimmune inflammatory diseases (e.g., long term allergic asthma control, or ankylosing spondylitis).

As shown in Example 4, an increase of HLA-H expression is positively associated with higher tumor stage. This indicates that clinically more aggressive tumors become resistant to chemotherapy by increasing HLA-H expression. Determining the amount of the nucleic acid molecule as defined in item (I)(g) of the first aspect of the invention and/or the protein or peptide as defined in connection with the first aspect of the invention can thus be used to determine the non-efficacy of a tumor treatment in a subject. Since HLA-H helps the tumor to escape the anti-tumor treatment by its immunosuppressive function, the nucleic acid molecule as defined in item (I)(g) of the first aspect of the invention and/or the protein or peptide as defined in connection with the first aspect of the invention can likewise be used for monitoring the non-efficacy of a immunosuppressive therapy.

In accordance with a preferred embodiment of all aspects of the invention relating to a tumor as described herein above, the tumor is cancer.

Cancer is an abnormal malignant new growth of tissue that possesses no physiological function and arises from uncontrolled usually rapid cellular proliferation.

In accordance with a more preferred embodiment of all aspects of the invention relating to a tumor as described herein above, the cancer is selected from the group consisting of breast cancer, ovarian cancer, endometrial cancer, vaginal cancer, vulva cancer, bladder cancer, salivary gland cancer, pancreatic cancer, thyroid cancer, kidney cancer, lung cancer, cancer concerning the upper gastrointestinal tract, colon cancer, colorectal cancer, prostate cancer, squamous-cell carcinoma of the head and neck, cervical cancer, glioblastomas, malignant ascites, lymphomas and leukemias.

In accordance with a more preferred embodiment of all aspects of the invention relating to a tumor as described herein above, the cancer is bladder cancer or a gynecologic cancer.

In accordance with a further more preferred embodiment of all aspects of the invention relating to a tumor as described herein above, the cancer is breast cancer or ovarian cancer.

Ovarian cancer is preferred since it is examiner in Example 4.

In accordance with a preferred embodiment of all aspects of the invention as described herein above, the sample is a body fluid or a tissue sample from an organ.

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The figures show.

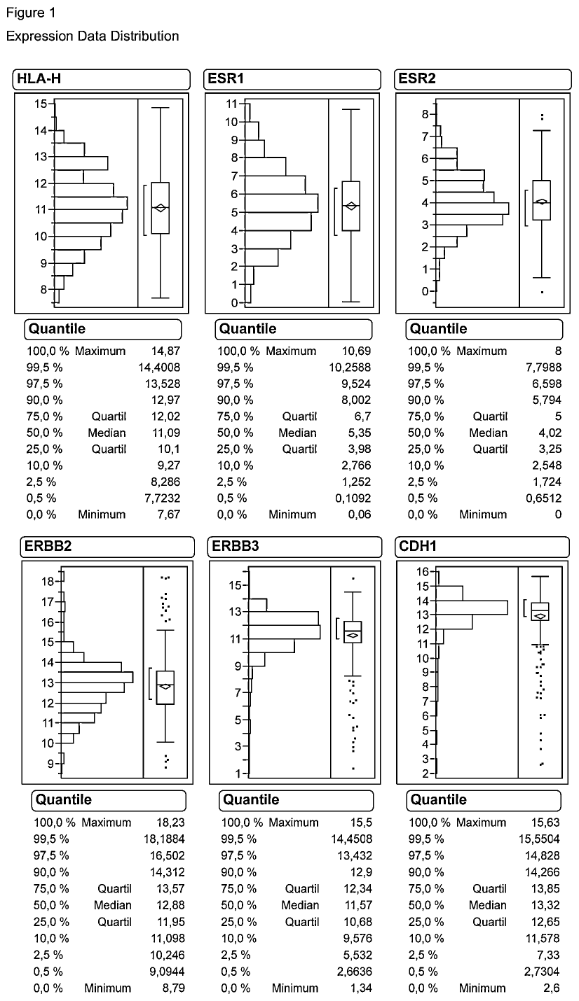

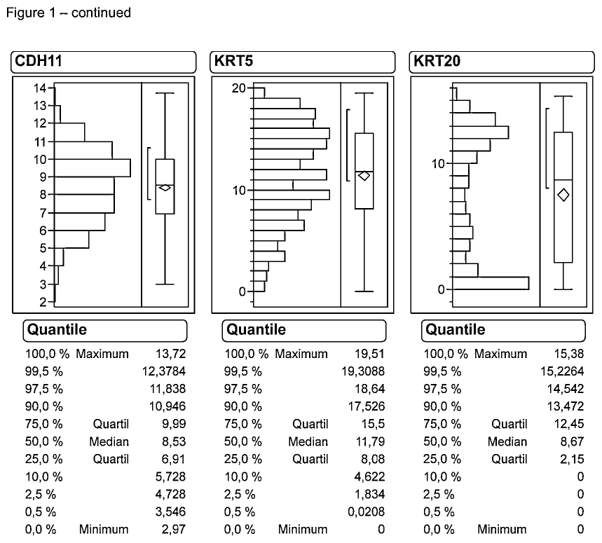

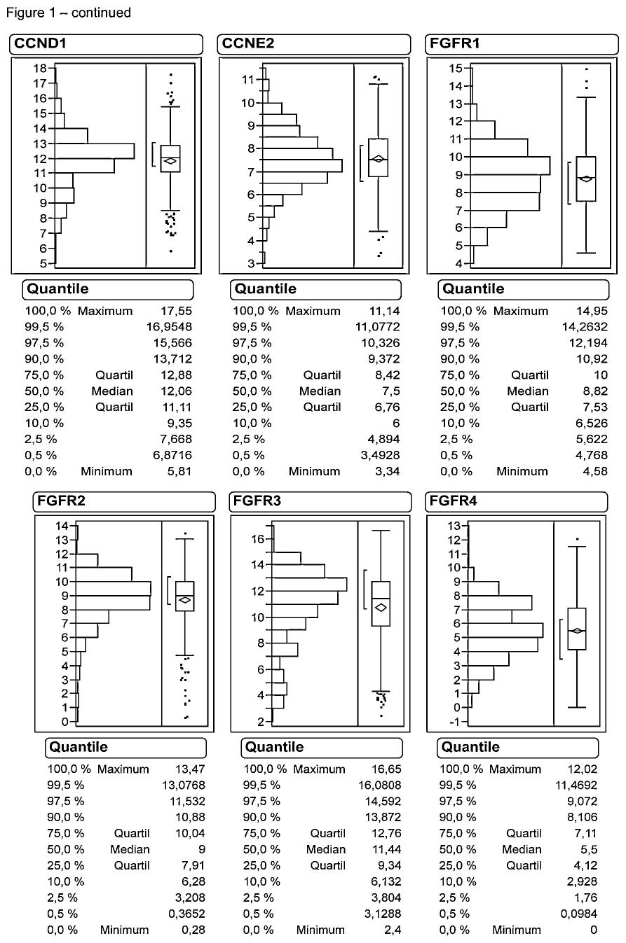

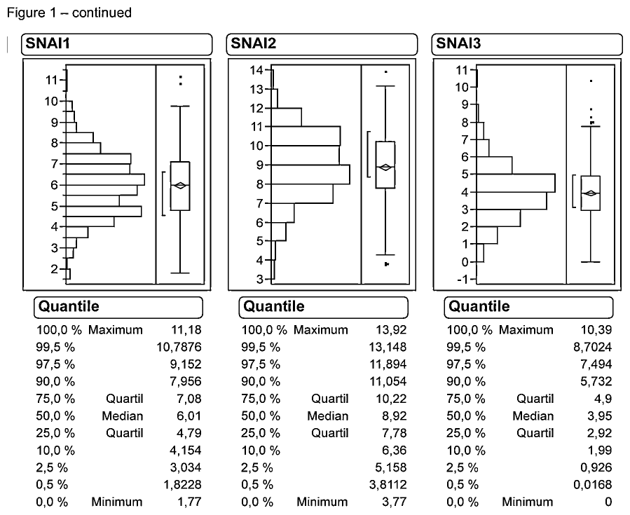

FIG. 1. Data distribution of HLA-H gene expression and Subtyping candidate gens in bladder cancer including FGF receptors as determined by RNAseq in muscle invasive bladder cancer samples (n=407).

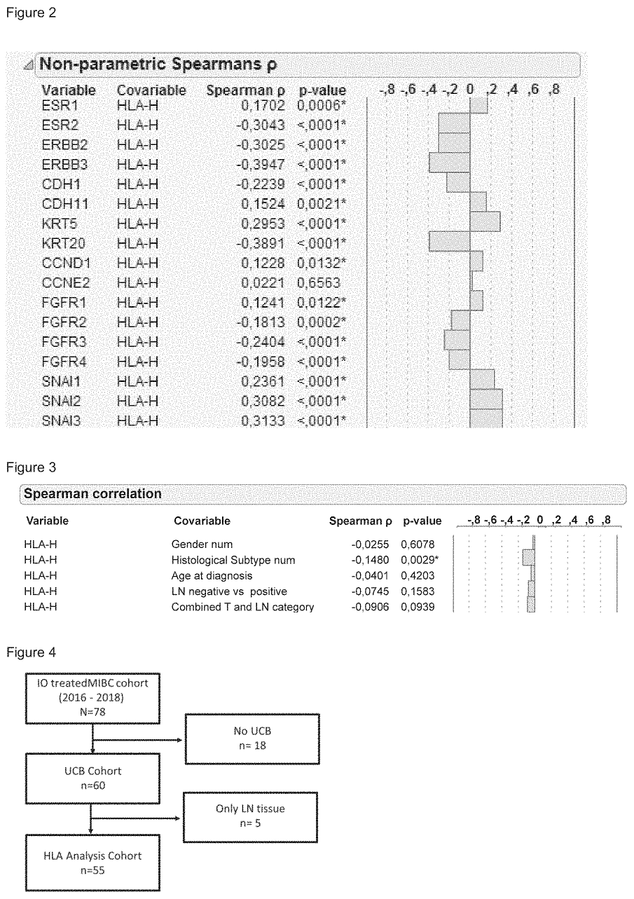

FIG. 2. Spearman correlation of HLA-H expression with luminal, basal and EMT markers as determined by RNAseq in muscle invasive bladder cancer samples (n=407).

FIG. 3. Spearman correlation of HLA-H expression with clinical variables such as gender, histological subtype, age and lymph node status in muscle invasive bladder cancer samples (n=407).

FIG. 4. Consort Diagram of advanced or metastatic urothelial cancer cohort. After exclusion of FFPE blocks with insufficient and/or lymphnode tissues, tissues of 55 patients were available for analysis.

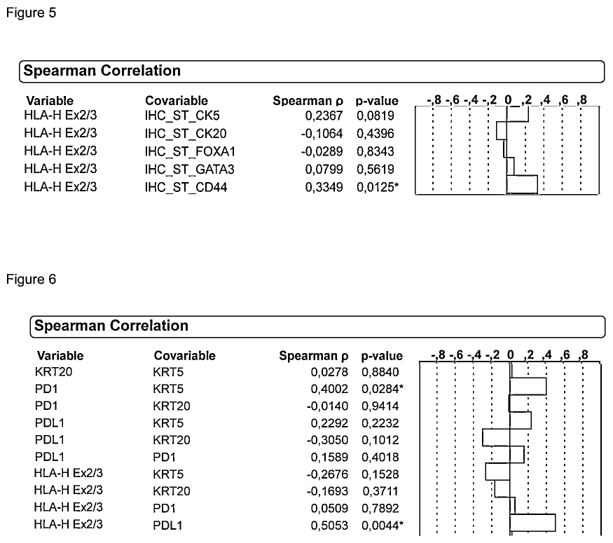

FIG. 5. Spearman correlation of HLA-H expression as determined at the Exon2/Exon 3 boundary with subtyping markers determined by standard IHC panels (n=55).

FIG. 6. Spearman correlation of HLA-H expression as determined at the Exon2/Exon 3 boundary with subtyping markers determined by RT-qPCR (n=55).

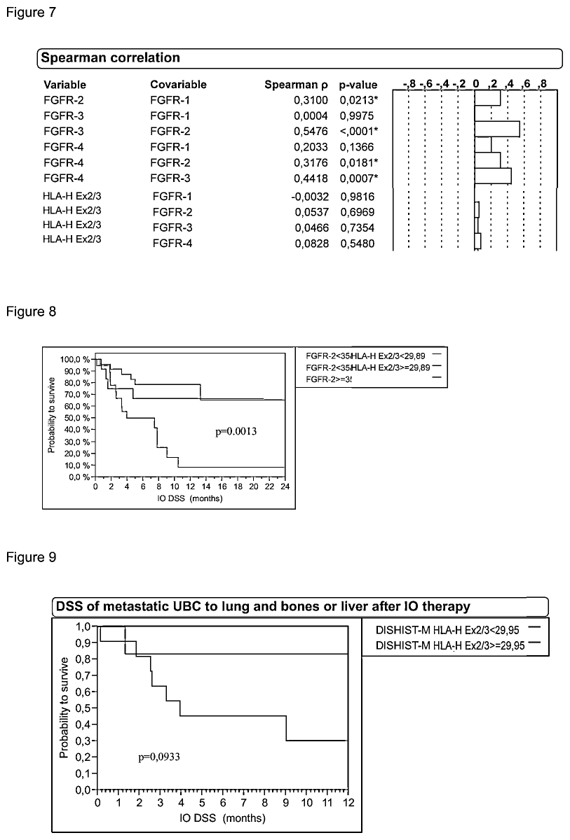

FIG. 7. Spearman correlation of HLA-H expression as determined at the Exon2/Exon 3 with FGFR target gene expression as determined by RT-qPCR (n=55).

FIG. 8. Kaplan Meier Plot displaying disease specific survival (DSS) probability from muscle invasive bladder cancer patients based on stratification by HLA-H Ex2/3 expression as quantified by RT-qPCR assay. Relative mRNA expression is determined by the 40-DCT method using CALM2 as reference gene.

FIG. 9. Kaplan Meier Plot displaying disease specific survival (DSS) probability from muscle invasive bladder cancer patients having metastasized to lung and bones or liver (n=17) based on stratification by HLA-H exon 2/3 expression as quantified by RT-qPCR assay. Relative mRNA expression is determined by the 40-DCT method using CALM2 as reference gene.

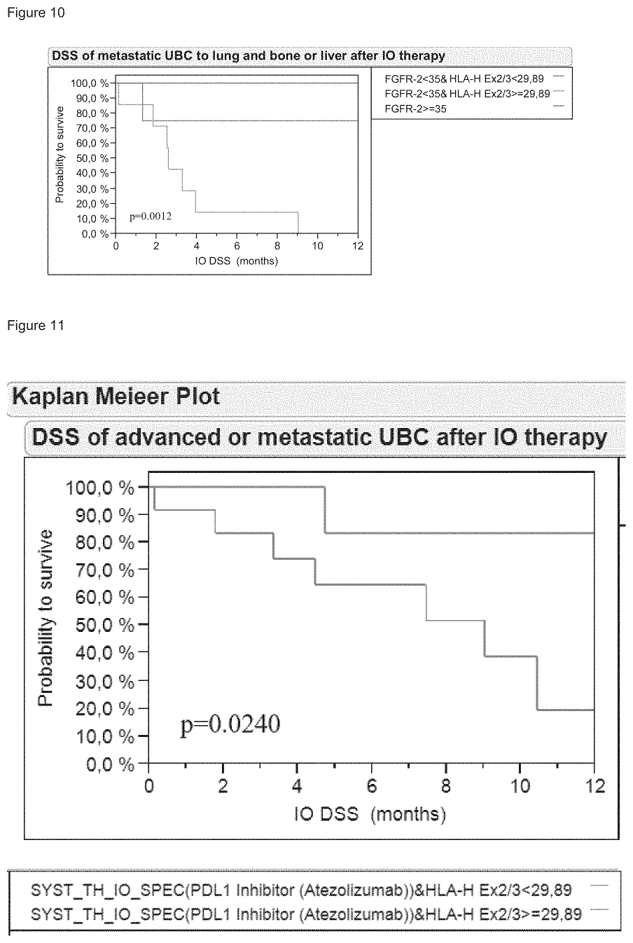

FIG. 10. Kaplan Meier Plot displaying disease specific survival (DSS) probability from muscle invasive bladder cancer patients having metastasized to lung and bones or liver (n=17) based on stratification by HLA-H exon 2/3 and FGFR2 expression as quantified by RT-qPCR assay. Relative mRNA expression is determined by the 40-DCT method using CALM2 as reference gene.

FIG. 11. Kaplan Meier Plot displaying disease specific survival (DSS) probability from chemotherapy resistant, muscle invasive bladder cancer patients having been treated with the PD-L1 specific checkpoint inhibitor (n=18) based on stratification by HLA-H exon 2/3 as quantified by RT-qPCR assay. Relative mRNA expression is determined by the 40-DCT method using CALM2 as reference gene.

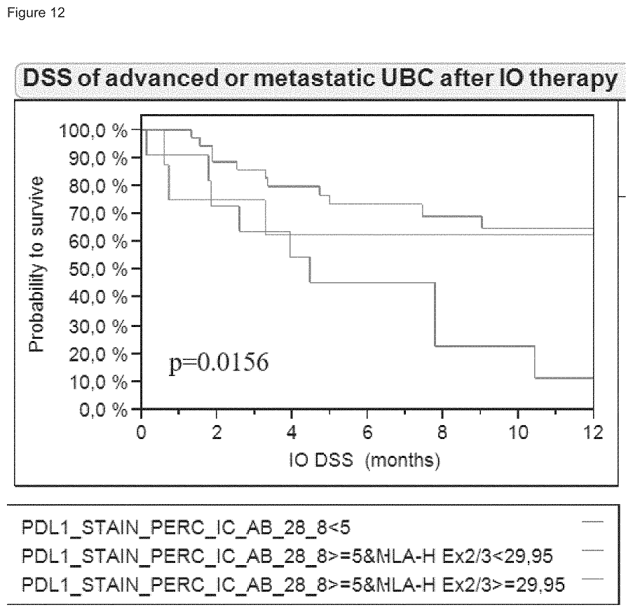

FIG. 12. Kaplan Meier Plot displaying disease specific survival (DSS) probability from chemotherapy resistant, muscle invasive bladder cancer patients based on stratified by IHC based PD-L1 staining on tumor infiltrating immune cells and by HLA-H exon 2/3 as quantified by RT-qPCR assay as indicated. Relative mRNA expression is determined by the 40-DCT method using CALM2 as reference gene.

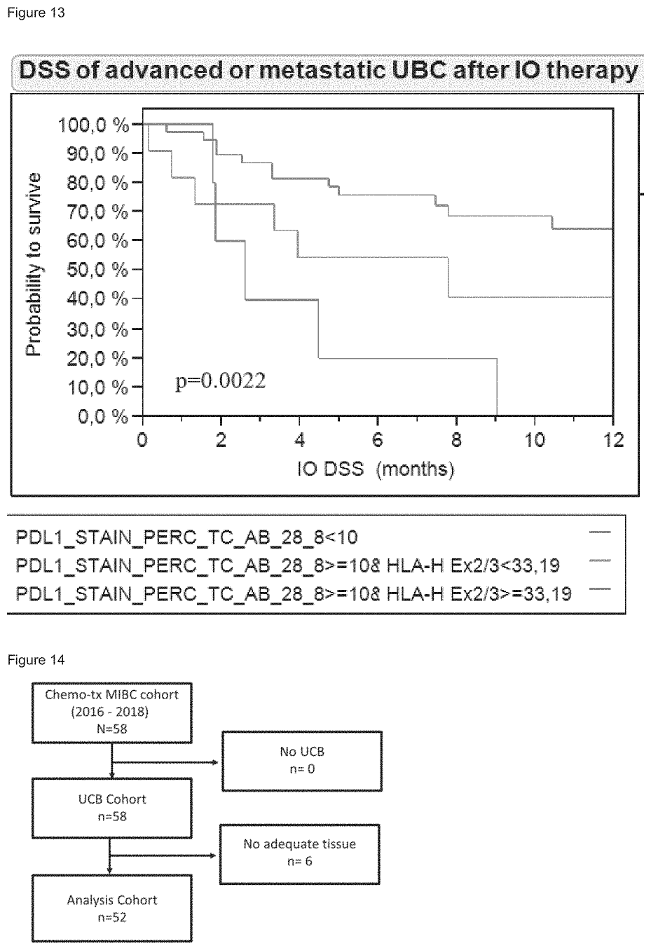

FIG. 13. Kaplan Meier Plot displaying disease specific survival (DSS) probability from chemotherapy resistant, muscle invasive bladder cancer patients based on stratified by IHC based PD-L1 staining on tumor cells and by HLA-H exon 2/3 as quantified by RT-qPCR assay as indicated. Relative mRNA expression is determined by the 40-DCT method using CALM2 as reference gene

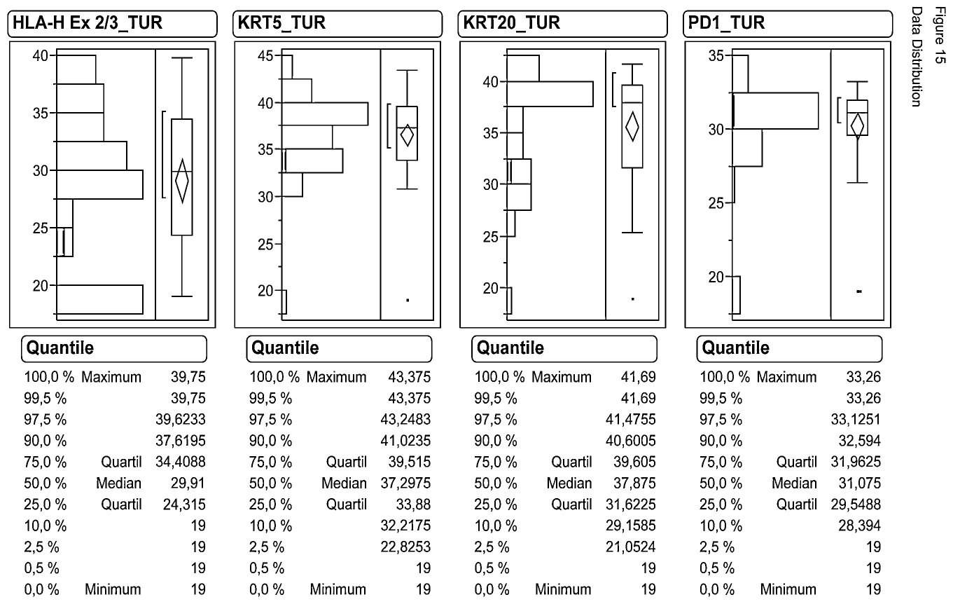

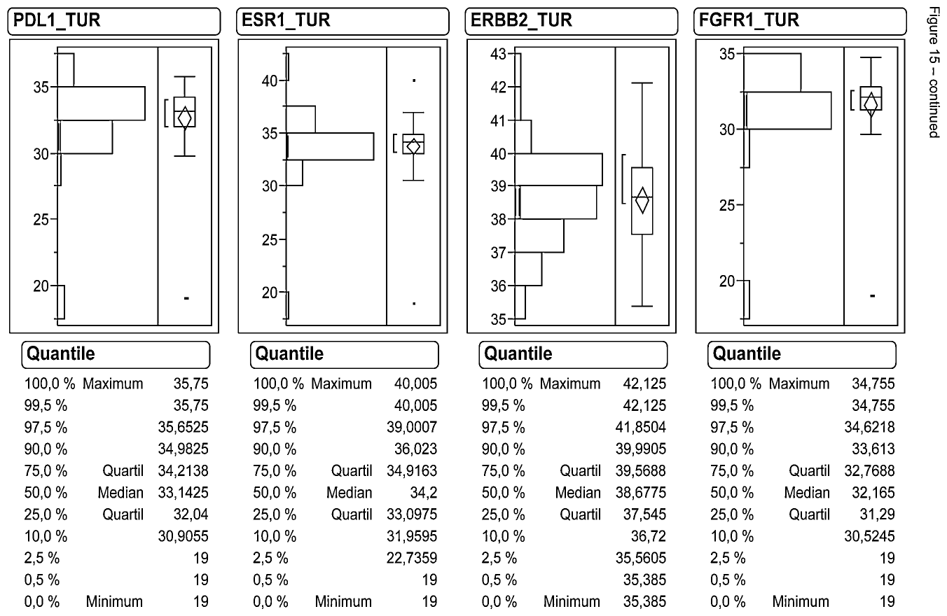

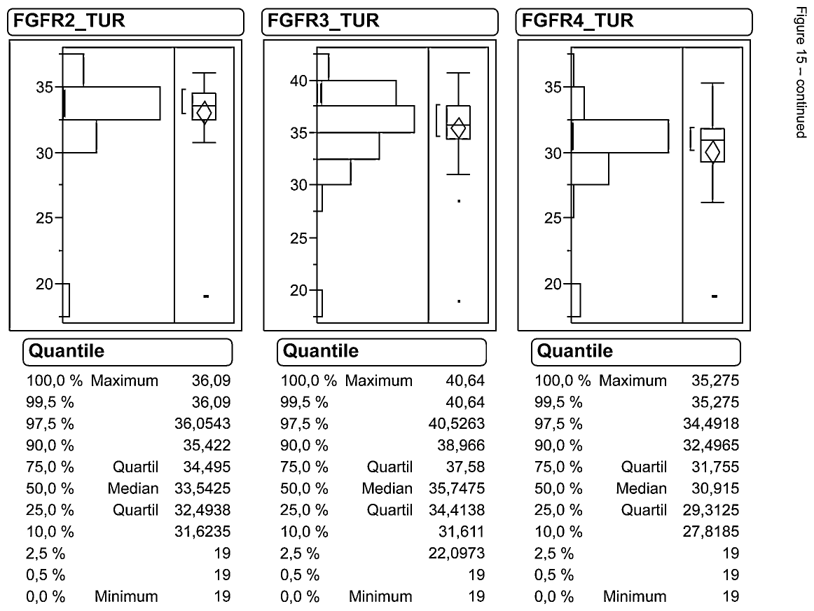

FIG. 14: Consort Diagram of advanced or metastatic urothelial cancer cohort. After exclusion of matched FFPE block pairs with insufficient tissues pre and post neoadjuvant chemotherapy 52 patients were available for analysis.

FIG. 15. Data distribution of HLA-H gene expression and subtyping markers as well as drug targets in bladder cancer as determined by RT-qPCR in therapy naïve TUR biopsies of muscle invasive bladder cancer samples (n=52).

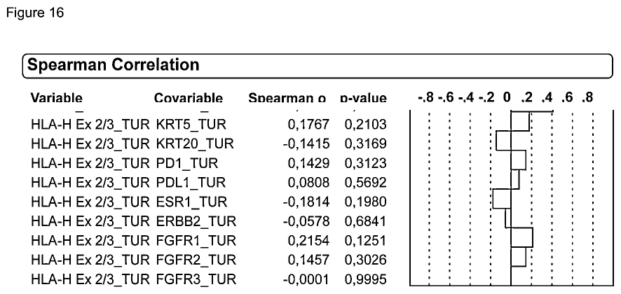

FIG. 16. Spearman correlation of HLA-H expression as determined at the Exon2/Exon 3 boundary with subtyping markers determined by RT-qPCR (n=52).

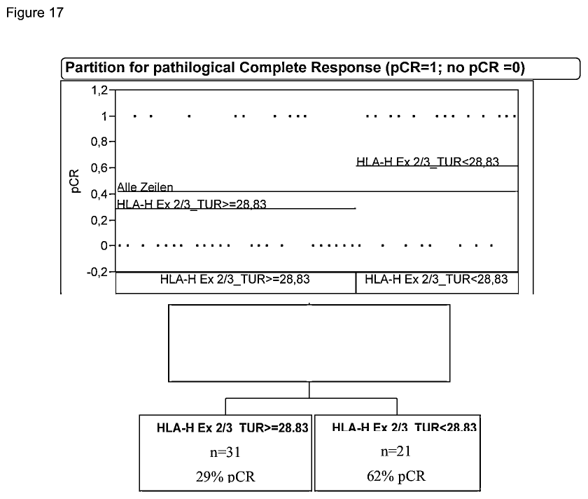

FIG. 17. Partition test for pathological complete response (pCR) after 3 cycles of neoadjuvant chemotherapy (Gem/Cis) based on HLA-H Exon 2/Exon 3 mRNA expression as determined in TUR biopsy samples by RT-qPCR (n=52). The numbers of patients in each group and percentages of respective chemotherapy responses are depicted.

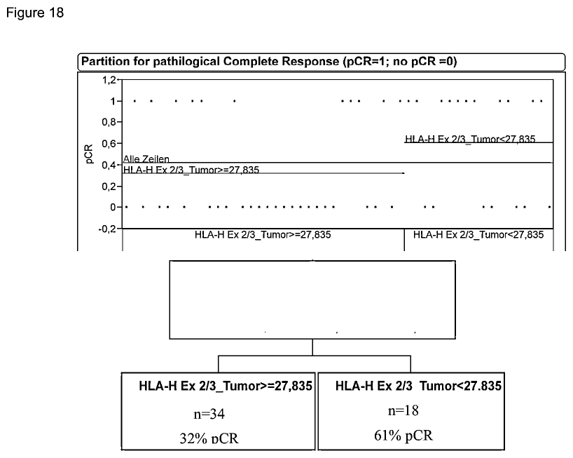

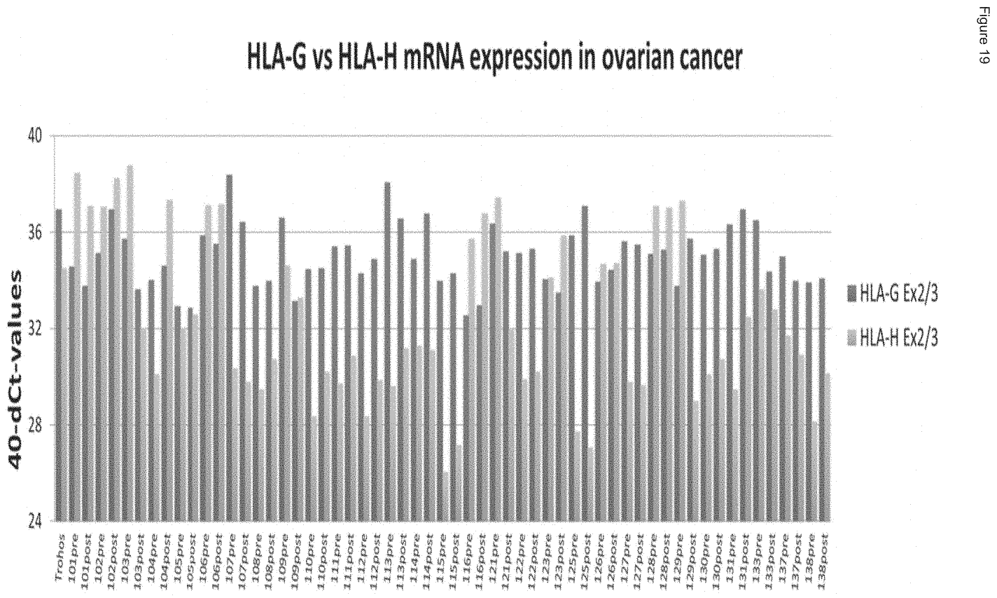

FIG. 18. Partition test for pathological complete response (pCR) after 3 cycles of neoadjuvant chemotherapy (Gem/Cis) based on HLA-H Exon 2/Exon 3 mRNA expression as determined in cystectomy samples after chemotherapy by RT-qPCR (n=52). The numbers of patients in each group and percentages of respective chemotherapy responses are depicted.

FIG. 19. HLA-H Exon 2/Exon 3 mRNA expression and HLA-G Exon 2/Exon 3 mRNA expression pre and post neoadjuvant chemotherapy as quantified by RT-qPCR assays. Relative mRNA expression is determined by the 40-DCT method using CALM2 as reference gene. The higher the 40-DCT value, the higher the gene expression. Due to exponential nature of the PCR method each increase by only 1 means a doubling of gene expression. An increase of 3 DCT values means an 8 fold increase of HLA-H mRNA expression.

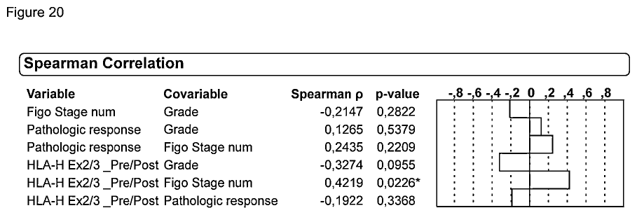

FIG. 20. Spearman correlation of HLA-H Exon 2/Exon 3 mRNA changes as determined by RT-qPCR in pre- and post chemotherapy specimen with clinicopathological variable and response parameters (n=27).

The examples illustrate the invention.

### Example 1: Spearman Correlation of HLA-H Expression with Bladder Cancer Candidate Genes

The first cohort comprised 407 cases that were accumulated by the Cancer Genome Atlas (TOGA) Research Network from 19 sites (Network CGAR. Comprehensive molecular characterization of urothelial bladder carcinoma. Nature. 2014; 507(7492):315-22. doi: 10.1038/nature12965. PubMed PMID: 24476821; PubMed Central PMCID: PMCPMC3962515). RNA-Seq (HiSeq) was used for whole genome analysis in tumor samples as previously described. This way, four molecular subtypes have been defined based on mRNA expression patterns (i.e. TCGA subtypes): the subtypes I and II are described as luminal-like, with subtype I being defined by FGFR3 alterations and elevated FGFR3 expression, while subtype II is characterized by ERBB2 mutations and estrogen receptor beta (ESR2) enrichment. Subtypes III and IV are described as basal-like, defined by increased expression of epithelial lineage genes and stem/progenitor cytokeratines. The clinical and molecular data are publicly available at the cBioPortal for Cancer Genomics website (http://www.cbioportal.org/study?id=blca_tcga#clinical). The data sets were downloaded and validated. Since most patients in the TCGA cohort had muscle-invasive bladder cancer (MIBC), patients with stages T0 and T1 were excluded from the analysis. Aside from 10 patients who received radiotherapy, the exact treatment modality was not documented. Therefore, we used a documented pN status as a surrogate for surgical therapy. All patients with pNX were excluded from the analysis, as were patients who received neoadjuvant therapy, definitive radiotherapy or had documented metastases. We analyzed the association between HLA gene expression with selected candidate markers for elevant tumorbiological motifs such as molecular subtype (e.g. KRT5, KRT20), hormone axis (e.g. ESR1, ESR2), adhesion motif (e.g. CDH1, CDH2, CDH11), cell cycle genes (e.g. CCND1, CCNE2), subtype specific target genes (e.g. ERBBs and FGFRs).

As depicted in FIG. 1 the substantial amounts of HLA-H “pseudogene” mRNA could be determined by RNA seq reaching similar amounts as well defined genes such as ERBB2, CDH1 and FGFR2 and FGFR3.

Next we analyzed whether the HLA-H expression was associated with other bladder cancer candidate genes for subtyping and drug targeting. As depicted in FIG. 2 the mRNA expression of HLA-H was negatively associated with the luminal subtype markers ESR2, ERBB2, ERBB3, CDH1 and KRT 20 (p<0.0001), but positively associated with candidate genes of basal subtype marker KRT5 (Spearman rho 0.2953; p<0.0001) and the Epithelial-Mesenchymal-Transition (EMT) markers SNAI1-3. Furthermore, the mRNA Expression was negatively associated with FGFR2, FGFR3 and FGFR4 with moderates correlation coefficients ranging between Spearman rho −0.1813 to −0.2404 (p=0.0002, p<0.0001 and p<0.0001; respectively).

We then analyzed the association of HLA-H mRNA expression in MIBC with tumor stage, tumor grade according to the nodal status, age, gender and histological subtye (papillary vs non papillary). As depicted in FIG. 3 HLA-H mRNA expression was significantly associated with non papillary histological subtype (p=0.0029). With regard to other clinical variables such as gender, age, or lymphnode status no significant association could be found.

### Example 2: Determination of HLA mRNA Expression Levels by Reverse Transcription (RT) Quantitative PCR (RT-qPCR) in a Muscle Invasive Bladder Cancer Patient Cohort Treated with Checkpoint Inhibitor Drug Upon Progression after Failure of Cystectomy Followed by Chemotherapy Regimen

Paraffin embedded tumor tissue samples surgical specimen from radical cystectomies and the corresponding transurethral resections were obtained from 78 patients suffering advanced urothelial cancer with centrally confirmed MIBC (pT2-T4). Patients were treated with therapeutic antibodies targeting the immunemodulatory check point targets PD-1 or PD-L1. Ethical approval was obtained from all participating centers and all patients gave informed consent. For RNA extraction from FFPE tissue, a single 10 μm curl was processed according to a commercially available bead-based extraction method (XTRAKT kit; STRATIFYER Molecular Pathology GmbH, Cologne, Germany). In brief, a lysis buffer was used to liquefy FFPE tissue slices while melting of paraffin was carried out in a thermo-mixer. Tissue lysis was accomplished with a proteinase K solution. Thereafter, lysates were admixed with germanium-coated magnetic particles in the presence of special buffers, which promote the binding of nucleic acids. Purification was carried out by means of consecutive cycles of mixing, magnetization, centrifugation and removal of contaminants. RNA was eluated with 100 μl elution buffer and RNA eluates were then stored at −80° C. until use. All extracts were tested for sufficient high quality RNA content by quantification with real time PCR (RT-qPCR) of the constitutively expressed gene Calmodulin 2 gene (CALM2) which is known as a stable reference/housekeeper gene. Specimens with a low CALM2 expression were excluded.

For a detailed analysis of gene expression by RT-q PCR methods, primers flanking the region of interest and a fluorescently labeled probe hybridizing in-between were utilized. Target-specific primers and probes were selected using the NCBI primer designing tool (www.ncbi.nlm.nih.go). RNA-specific primer/probe sequences were used to enable RNA-specific measurements by locating primer/probe sequences across exon/exon boundaries. Furthermore, primers/probes were selected not to bind to sequence regions with known polymorphisms (SNPs). In case multiple isoforms of the same gene existed, primers were selected to amplify all relevant or selected splice variants as appropriate. All primer pairs were checked for specificity by conventional PCR reactions. After further optimization of the primers/probes, the primers and probes listed in table 1 gave the best results. These primers/probes are superior to primers/probes known from the prior art, e.g., in terms of specificity and amplification efficiency. TaqMan® validation experiments were performed showing that the efficiencies of the target and the control amplifications were approximately equal, which is a prerequisite for the relative quantification of gene expression by the comparative ACT method.

TABLE 1
Used primers and probes for HLA mRNA quantitation

| Gen | For_Primer | Probe | Rev-Primer |
|---|---|---|---|
| HLA-G Ex 2/3 | CCGAACCCTCTTCCTGCTGC | CGAGACCTGGGCGGGCTCCC | GCGCTGAAATACCTCATGGA |
| HLA-H Ex 2/3 | GAGAGAACCTGCGGATCGC | AGCGAGGGCGGTTCTCACACCATG | CCACGTCGCAGCCATACAT |
| KRT5 | CGCCACTTACCGCAAGCT | TGGAGGGCGAGGAATGCAGACTCA | ACAGAGATGTTGACTGGTCCAA |
| | | | CTC |
| KRT20 | GCGACTACAGTGCATATTACAGA | TTGAAGAGCTGCGAAGTCAGATTAA | CACACCGAGCATTTTGCAGTT |
| | CAA | GGATGCT | |
| PD-1 | GGCCAGCCCCTGAAGGA | ACCCCTCAGCCGTGCCTGTGTTC | GGAAATCCAGCTCCCCATAGTC |
| PDL1 | CAAAGTGATACACATTTGGAGGA | TGGCATCCAAGATACAAACTCAA | TTGAAGATCAGAAGTTCCAATGCT |
| | GACGTAA | | |
| CALM2 | GAGCGAGCTGAGTGGTTGTG | TCGCGTCTCGGAAACCGGTAGC | AGTCAGTTGGTCAGCCATGCT |
| ESR1 | GCCAAATTGTGTTTGATGGATTAA | ATGCCCTTTTGCCGATGCA | GACAAAACCGAGTCACATCAGTA |
| | | | ATAG |
| ERBB1 | TCTGGACGTGCCAGTGTGAA | AGGCCAAGTCCGCAGAAGCCCT | CCTGCTCCCTGAGGACACAT |
| MMP28 | TGCCTAACGCTAGCCTTCAGA | CCTCCTAATGAAGAAAGGAAACCT | GGCTTAAGTCCCCAGCTTGA |
| | | AATCAGACCCC | |
| HPRT1 | GGGTGTTTATTCCTCATGGACTA | TGGACAGGACTGAACGTCTTGCTC | GGCCTCCCATCTCCTTCATC |
| | ATT | GAG | |

The final cohort consisted of 55 cystectomy-specimens from primary tumor tissues. Comparative analysis was performed with TUR biopsies and FFPE samples from metastatic lesions or simultaneous upper tract tumors (UTUC) as far as available.

Gene specific TaqMan-based Primer/Probe sets for the assessment of the mRNA expression of HLA-H were designed and tested for sensitivity and specificity. Immuno-histochemical staining of CK5, CK20, GATA3, FOXA1, CD44 was performed to determine whether HLA-H expression is associated with bladder cancer subtypes defined by the international classification consensus. Representative FFPE blocks with at least 50% tumor content (minimal tumor size 5×5 mm), well delimited invasion borders, and without necrotic regions or granulomatous inflammation were selected. All IHC stainings were performed and read on whole slide sections as follows. Immuno-histochemical stains were performed with 4 μm tissue sections using an automated Ventana Benchmark Ultra autostainer (Ventana, Tucson, Ariz., USA).

Briefly, tissue sections were deparaffinized, antigens retrieved by heat treatment in a Tris/Borate/EDTA solution pH 8.4 (Ventana) and endogenous peroxidase was blocked with 1% H2O2. PDL1 immuno-staining was performed with a commercially available assay kit from DAKO adapted and validated for the Ventana platform (DAKO 28-8, DAKO, USA). CK5 (Clone XM26, monoclonal mouse, DiagnosticBioSystems®, dilution 1:50), CK20 (Clone Ks 20.8, mouse monoclonal, DAKO®, dilution 1:50), GATA3 (clone L50-823, mouse, monoclonal, DCS®, dilution 1:100), CD44 (clone DF1485, mouse, monoclonal, Dako®, dilution 1:50) and FOXA1 (clone AB55178, mouse, monoclonal, Abcam, dilution 1:2000) were immuno-stained according to a standardized and accredited staining protocol using a Benchmark Ultra autostainer (Ventana, USA). Revelation was performed using the ultraVIEW™ DAB systems (Ventana). All tissue sections were counterstained with hematoxylin II/Mayer's hematoxylin (Ventana).

As depicted in FIG. 5, when determining bladder cancer subtypes in this cohort of advanced, chemotherapy resistant tumors by IHC the mRNA expression of HLA-H was particularly associated with the stem cell marker CD44 (Spearman rho 0.3349; p=0.0125), which is more frequent in KRT5 positive, basal bladder cancer.

Molecular subtyping of bladder cancer has become one major tool to look for stratification of tumors into hormone dependent luminal tumors with less immune cell infiltration and basal or inflamed subtypes having higher frequencies of tumor infiltrates, which impacts survival in the non 10 treated setting of muscle invasive bladder cancer. (Pfannstiel C, Strissel P L, Chiappinelli K B, Sikic D, Wach S, Wirtz R M, Wullweber A, Taubert H, Breyer J, Otto W, Worst T, Burger M, Wullich B, Bolenz C, Fuhrich N, Geppert C I, Weyerer V, Stoehr R, Bertz S, Keck B, Erlmeier F, Erben P, Hartmann A, Strick R, Eckstein M; BRIDGE Consortium, Germany; BRIDGE Consortium, Germany; BRIDGE Consortium, Germany; BRIDGE Consortium, Germany. The Tumor Immune Microenvironment Drives a Prognostic Relevance That Correlates with Bladder Cancer Subtypes. Cancer Immunol Res. 2019 June; 7(6):923-938. doi: 10.1158/2326-6066.CIR-18-0758. Epub 2019 Apr. 15).

Therefore it was of great interest to determine the correlation of HLA-H mRNA expression on basis of molecular subtyping and targets of immune checkpoint targets. In addition, to further elucidate potential effects of splicing events occurring with regard to this formerly anticipated “pseudogene”, different primer probe sets were designed at distinct Exon/Exon boundaries.

As depicted in FIG. 6, the mRNA expression of standard markers for molecular subtyping within the chemotherapy resistant muscle invasive bladder cancer cohort revealed a dominance of PD1 positive immune cell infiltrates in the basal, KRT5 positive subtype. However, there was no negative correlation of KRT5 and KRT20 indicating that this selected, therapy resistant tumor population is heterogenous. Interestingly a diversity of HLA-H mRNA's became apparent, when quantifying the exon/exon boundary at exon 2/3 coding for the extracellular part of HLA-H containing the Exon 2/Exon 3 boundary strongly being associated with high PD-L1 expression (Spearman rho 0.5053; p=0.0044), but not with PD1 expression and dominating in KRT5 & KRT20 negative subpopulations of bladder cancer.

Correlation of HLA-H splice variants with mRNA expression of FGFR1, FGFR2, FGFR3 and FGFR4 revealed, isoforms containing Exon 2/Exon 3 were not associated with FGFR mRNA expression (see FIG. 7). Moreover, it could be determined that FGFR family expression is predictive for survival of advanced or metastatic bladder cancer, with FGFR2 being associated with good outcome, while mutations, fusions or overexpression of FGFR3 is associated with adverse outcome despite immuneoncological treatment with checkpoint inhibitory drugs as has been filed elsewhere (EP19168923.1; Applicant STRATIFYER Molecular Pathology GmbH).

This prompted us to investigate whether the interaction of HLA-H Exon 2/Exon 3 mRNA expression, which is associated with PD-L1 mRNA expression, does have an impact on survival in FGFR2 negative tumors having higher risk of cancer specific survival despite anti-PD-L1 and PD-1 treatment. As depicted in FIG. 8 advanced or metastatic urothelial cancer patients exhibited significantly worse disease specific survival as determined from start of first, second or third line treatment with immunemodulatory checkpoint inhibitors such as atezolizumab, pembrolizumab or nivolumab if the primary, FGFR2 negative tumor does express HLA-H as determined by RNA specific RT-qPCR. The survival probability of patients with FGFR2 negative tumors expressing high levels of HLA-H Ex2/3 was at 10% after 2 years, while patients with FGFR2 negative tumors not expressing HLA-H Ex2/3 had a survival probability of 65% after 2 years (p=0.0013).

To further elucidate the relevance of HLA-H expression on survival after 10 therapy the analysis was specified by also taking the primary metastatic site into account. This is based on initial findings that 10 therapy has differential effects depending on the site of metastasis and being less effective with e.g. visceral metastasis into the liver, probably due to the fact that PD1 positive T-cells are being excluded from the liver in metastatic urothelial cancer patients independent of classical checkpoint mechanisms (Eckstein M, Sikic D, Strissel P L, Erlmeier F. Evolution of PD-1 and PD-L1 Gene and Protein Expression in Primary Tumors and Corresponding Liver Metastases of Metastatic Bladder Cancer., Eur Urology 2018). Therefore the patients were grouped according to the first manifestation of metastasis with local advancement, locoregional lymph nodes or extraregional retroperitoneal lymph nodes being categorized as 0 or 0.5, respectively, while dissemination into the bones, liver, lung, lung and bone or lung and liver were categorized with increasing indices (1, 2, 3, 4, 5; respectively). For this analysis 55 datasets from primary tumor tissues with sufficient clinical date and primary tumor tissue material were available, with 20 patients having local advancement or lymph node metastasis, while 18 patients had initially metastasized to bone or liver and 17 patients having metastasized with lung involvement either as singular site or in combination with bone or liver involvement, while all of them had been treated with 10 drugs and predominantly >1^{st} line setting (74%).

Interestingly the adverse outcome upon HLA-H expression was particularly striking in the metastasized situation. As depicted in FIG. 9 high HLA-H Exon 2/3 mRNA expression (>=29.95) was associated with inferior disease specific survival with 6 HLA-H Exon 2/3 positive patients having a survival probability of only 30% after 1 year, while 11 HLA-H Exon 2/3 negative patients had a survival probability of 80% after 1 year, which only trended to be significant due to early crossing of the curves which corrupts log rank tests.

The predictive value could be increased, when the FGFR2 expression was taken into account. As depicted in FIG. 10 high HLA-H Exon 2/3 mRNA expression (>=29.95) in FGFR negative patients was associated with inferior disease specific survival with HLA-H Exon 2/3 positive patients having a survival probability of only 0% after 1 year, while HLA-H Exon 2/3 & FGFR2 negative patients had a survival probability of 70% after 1 year. Metastatic bladder cancer patients having high FGFR2 expression had 100% disease specific survival after 1 year (p=0.0012).

As described above it was found that the mRNA expression of HLA-H Exon 2/Exon 3 boundary was associated with PD-L1 mRNA expression (Spearman rho 0.5053; p=0.0044). Therefore it seemed reasonable to stratify the whole patient cohort (advanced and metastasized; n=55) by the underlying principle of applicated immunemodulatory checkpoint treatment, i.e. whether a PD-1 or PD-L1 treatment had been applied. While pembrolizumab and nivolumab specifically affect the checkpoint by binding to PD-1 positive T-cells, atezolizumab binds to PD-L1 which is present on tumor cells or macrophages and additional cell sources, thereby potentially having a more pleiotrop effect. Importantly, when just looking at atezolizumab treated patients, the significance of HLA-H Exon 2/Exon 3 boundary expression became particularly evident. As depicted in FIG. 11 high HLA-H Exon 2/3 mRNA expression (>=29.89) was associated with inferior disease specific survival with HLA-H Exon 2/3 positive patients having a survival probability of only 10% after 1 year, while HLA-H Exon 2/3 negative patients had a survival probability of 80% after 1 year (p=0.0240).

To further elucidate the interaction between PD-L1 expression and HLA-H function and in view of pleiotropic effects the cohort of advanced and metastatic bladder cancer patients having being treated with immunemodulatory checkpoint inhibitors was stratified on basis of PD-L1 protein expression on immune cells (presumably macrophages) based on IVD tests for PD-L1 determination. Here a cut-off for positivity of 5% positive immune cells was chosen to exclude unspecific staining effect on macrophages based on basal peroxidase activity not related to the IHC detection system.

Interestingly, patients having lower frequency of PD-L1 positive tumor infiltrating immune cells had better survival, while patient having higher frequencies of PD-L1 positive tumor infiltrating immune cells had inferior survival, particularly when expressing HLA-H Exon 2/Exon 3 isoforms. As depicted in FIG. 12 high HLA-H Exon 2/3 mRNA expression (>=29.95) was associated with inferior disease specific survival in patients with more than 5% PD-L1 positive immune cells and with HLA-H Exon 2/3 positive patients having a survival probability of only 10% after 1 year, while HLA-H Exon 2/3 negative patients and patients having no or lower frequencies of PD-L1 positive immune cells had a survival probability of 60% after 1 year (p=0.0156).

However, knowing that there is an overlap in tumor tissues having PD-L1 positive immune cells versus PD-L1 positive tumor cells, we also examined the interaction of HLA-H expression with PD-L1 expression on tumor cells taking a standard cut-off for PD-L1 positive tumor cells of 10%.

Again PD-L1 positive patients having high HLA-H Exon 2/Exon 3 expression had inferior survival compared to those patients having lower HLA-H Exon 2/Exon 3 expression. As depicted in FIG. 13 high HLA-H Exon 2/3 mRNA expression (>=33.19) was associated with inferior disease specific survival in patients with at least 10% PD-L1 positive tumor cells and with HLA-H Exon 2/3 positive patients having a survival probability of only 0% after 1 year, while HLA-H Exon 2/3 negative patients had a survival probability of 40% after 1 year an patients having no or less than 10% PD-L1 positive tumor cells had a survival probability of 65% (p=0.0022).

### Example 3: Determination of HLA-H mRNA Expression Levels by Reverse Transcription (RT) Quantitative PCR (RT-qPCR) in a Muscle Invasive Bladder Cancer Patient Cohort Neoadjuvantly Treated with 3 Cycles of Gemcitabine/Cisplatinum Chemotherapy Regimen

Paraffin embedded tumor tissue samples surgical specimen from transurethral resections before chemotherapy and the corresponding radical cystectomies were obtained from 55 patients suffering advanced urothelial cancer with histologically confirmed MIBC, UICC stage II and III (cT2-3 and cN0 or cN+M0). Patients had underwent 3 neoadjuvant cycles of Gemcitabine 1250 mg/m2 (d1; d8) and Cisplatin 70 mg/m2 (d1) followed by radical cystectomy. Main inclusion criteria were the availability of pretreatment and posttreatment FFPE tissues. Main exclusion criterion was variant histology to obtain a homogenous bladder cancer cohort for analysis. Ethical approval was obtained from the participating center and all patients gave informed consent. For RNA extraction from FFPE tissue, a single 10 μm curl was processed according to a commercially available bead-based extraction method (XTRAKT kit; STRATIFYER Molecular Pathology GmbH, Cologne, Germany). In brief, a lysis buffer was used to liquefy FFPE tissue slices while melting of paraffin was carried out in a thermo-mixer. Tissue lysis was accomplished with a proteinase K solution. Thereafter, lysates were admixed with germanium-coated magnetic particles in the presence of special buffers, which promote the binding of nucleic acids. Purification was carried out by means of consecutive cycles of mixing, magnetization, centrifugation and removal of contaminants. RNA was eluated with 100 μl elution buffer and RNA eluates were then stored at −80° C. until use. All extracts were tested for sufficient high quality RNA content by quantification of the constitutively expressed gene Calmodulin 2 gene (CALM2) which is known as a stable reference/housekeeper gene. Specimens with a low CALM2 expression were excluded. The final cohort consisted of 52 cystectomy-specimens from primary tumor tissues after exclusion of 6 samples due to insufficient tumor material. Comparative analysis was performed with TUR biopsies and FFPE samples from cystectomy tissues.

For validation of the significance of HLA-H expression in bladder cancer apart from 10 therapies HLA-H has been quantitated in a similarly sized muscle invasive bladder cancer cohort, which has not yet been treated with any systemic therapy. As depicted in FIG. 15 substantial amounts of HLA-H “pseudogene” mRNA at the boundaries of Exon 2/Exon 3 could be determined by RT-qPCR. In addition the bladder cancer subtyping markers (KRT5, KRT20) and targets (PD-1, PD-L1, ESR1, ERBB2, FGFR1-4) were determined.

First the association of HLA-H with bladder cancer subtyping markers and targets was assessed in TUR biopsies of the neoadjuvant MIBC cohort. As depicted in FIG. 16 HLA-H expression as determined by RT-qPCR of the Exon2/Exon 3 boundary was positively associated with KRT5 and FGR1 expression, both of which are associated with the basal phenotype. In addition there were negative associations to KRT20 and ESR1, with KRT20 being a classical luminal marker. Therefore the associations reflected the initial finding in the mostly cystectomy samples of 400 MIBC, which also contained T1 tumors and variant histologies, though not reaching statistical significance potentially due to limited sample size.

Next the expression of HLA-H Exon 2/Exon 3 mRNA expression was correlated with response to three cycles of neoadjuvant chemotherapy (Gem/Cis) as described above with pathological complete response being defined as no vital tumor cell in the surgical specimen (cystectomy) after chemotherapy. In total 42% of patients achieved a pathological complete response. When stratifying the patients by partitioning testing based on the relative HLA-H Exon 2/Exon 3 mRNA expression, tumor expressing high levels of HLA-H accounting for 60% of all tumors had 2 fold reduced responsiveness to neoadjuvant chemotherapy, with HLA-H positive tumors responding in 29% of cases and HLA-H negative tumors responding in 62% of the cases. This indicates, that HLA-H expression determined in bladder cancer biopsy material before chemotherapy. In total 70% of tumors with high HLA-H expression did not respond to neoadjuvant chemotherapy.

Next the expression of HLA-H Exon 2/Exon 3 mRNA expression was determined in cystectomy specimen after three cycles of neoadjuvant chemotherapy (Gem/Cis). As depicted in FIG. 18 the HLA-H expression remained to be equally high in the non-responding tumors and an almost identical separation into tumors being resistant and having responded. In total 70% of tumors with high HLA-H expression did not respond to neoadjuvant chemotherapy.

### Example 4: Determination of HLA-H mRNA Expression Levels by Reverse Transcription (RT) Quantitative PCR (RT-qPCR) in Advanced Ovarian Cancer Patient Cohort Neoadjuvantly Treated with 6 Cycles of Paclitaxel/Cisplatinum Chemotherapy Regimen

Forty-five newly diagnosed patients with histologically confirmed FIGO stage III-IV epithelial ovarian or peritoneal carcinoma unsuitable for optimal upfront surgery and candidate for neo-adjuvant chemotherapy (said carcinoma also referred to herein below as ovarian cancer) were enrolled in the study between September 2004 and December 2007. Other inclusion criteria were age >18 years, haematological, renal, hepatic and cardiac function adequate for platinum-based chemotherapy. Exclusion criteria were a Karnofsky performance status (KPS) lower than 70%, a history of other malignancies and contraindications for surgery. The possibility of optimal debulking surgery was excluded at baseline by open laparoscopy. The initial study population of 45 patients was restricted to 35, after excluding nine patients whose biopsy samples were not adequate for the microarray analysis and one patient found to be ineligible because of diagnosis of peritoneal mesothelioma after histological revision. A standard regimen of carboplatin AUC 5 and paclitaxel 175 mg/m2 Q3 over 3 h every 3 weeks was administered as neo-adjuvant treatment for six cycles. In three patients older than 75 years and in one patient with poor performance status (KPS 70%), single-agent carboplatin was preferred to the combination chemotherapy.

Histopathological response was evaluated after surgery, with surgical samples analysis. To date, no histopathological criteria have been firmly established to describe treatment response after neo-adjuvant chemotherapy in ovarian cancer. According to the literature concerning response to primary chemotherapy in ovarian (Le et al. 2007, Sassen et al. 2007) and breast cancer (Ogston et al. 2003), as complete pathological response the absence of cancer cells in surgical specimens, and as very good partial remission the persistence of only small clusters (<1 cm) or individual cancer cells and no macroscopic residual after surgery was considered. Partial pathological remission was defined as a tumor burden reduction between 30% and 90% at surgery, while stable disease was defined as no tumor burden reduction or reduction lower than 30% at surgery, compared with initial diagnostic laparoscopy. Only patients with complete and very good partial remissions were considered as pathological responders, while all the other cases were considered as pathological non-responders.

For mRNA analysis, tissues collected were snap frozen and stored in liquid nitrogen until analysis. Approximately 20-100 mg of frozen ovarian tumor tissue was crushed in liquid nitrogen. RNA was extracted using commercial kits (Qiagen), RNA integrity was assessed on the Agilent 2100 Bioanalyzer (Agilent Technologies, Palo Alto, Calif., USA), cDNA was synthesized from 1 mg of total RNA using Invitrogen kits (Invitrogen Corp.) and by RT-qPCR using RNA specific primer Probe sets for HLA-H and subtyping markers as well as target genes as described above. Analysis was restricted to cases were pretreatment and posttreatment tissue samples were available, in total matched pair analysis was possible for 29 patients.

Similar to the situation in bladder cancer described above, substantial amounts of HLA-H mRNA could be detected in RNA extracts from pretreatment biopsies and posttreatment resectates of ovarian cancer patients, which reached comparable levels as HLA-G in the same tissues. However, the median mRNA expression for HLA-H Exon 2/Exon 3 was lower (40-DCT of 31.22) than for HLA-G Exon 2/Exon 3 (40-DCT of 35.00). As expected there was no close correlation for HLA-H and HLA-G expression.

When associating the difference of HLA-H Exon 2/Exon 3 mRNA expression pre and post chemotherapy with pathological response, there was an inverse relation between increase of HLA-H expression after chemotherapy and pathological response, both by Pearson correlation (r=−0.2290) and Spearman correlation (r=−0.1922). As depicted in FIG. 20, correlation with pathological stage revealed, that the increase of HLA-H expression is positively associated with higher Figo stage (Spearman r=0.4219; p=0.0226), indicating that clinically more aggressive tumors react to chemotherapy by increasing HLA-H expression. Interestingly this capability trended to be dominant in lower grade tumors (Spearman r=−0.3274; p=0.095).

1. A nucleic acid molecule, a vector, a host cell, or a protein or peptide, or combinations thereof for use as an immunosuppressant, as a tumor vaccine or as a pregnancy promoter wherein
(I) the nucleic acid molecule is
(a) encoding a polypeptide comprising or consisting of the amino acid sequence of SEQ ID NO: 1 or 54; or
(b) consisting of the nucleotide sequence of SEQ ID NO: 2; or
(c) encoding a polypeptide which is at least 95% identical to the amino acid sequence of SEQ ID NO: 1 or 54; or
(d) consisting of a nucleotide sequence which is 95% identical to the nucleotide sequence of SEQ ID NO: 2; or
(e) consisting of a nucleotide sequence which is degenerate with respect to the nucleic acid molecule of (d); or
(f) a fragment of the nucleic acid molecule of any one of (a) to (e), said fragment comprising at least 250 nucleotides, preferably at least 300 nucleotides, more preferably at least 450 nucleotides, and most preferably at least 600 nucleotides; or
(g) corresponding to the nucleic acid molecule of any one of (a) to (f), wherein T is replaced by U;
(II) the vector comprises the nucleic acid molecule of (I);
(III) the host cell is transformed, transduced or transfected with the vector of (II); and
(IV) the protein or peptide being encoded by the nucleic acid molecule of (I).

2. An inhibitor of the nucleic acid molecule as defined in claim 1 and/or a binding molecule of the protein as defined in claim 1, preferably an inhibitor of the protein as defined in claim 1 for use as an immunoactivator, preferably for use in the treatment of a tumor.

3. The binding molecule, preferably the inhibitor of claim 2, wherein
(I) the inhibitor of the nucleic acid molecule is selected from a small molecule, an aptamer, a siRNA, a shRNA, a miRNA, a ribozyme, an antisense nucleic acid molecule, a CRISPR-Cas9-based construct, a CRISPR-Cpf1-based construct, a meganuclease, a zinc finger nuclease, and a transcription activator-like (TAL) effector (TALE) nuclease, and/or
(II) the binding molecule of the protein, preferably the inhibitor of the protein is selected from a small molecule, an antibody or antibody mimetic, an aptamer, wherein the antibody mimetic is preferably selected from affibodies, adnectins, anticalins, DARPins, avimers, nanofitins, affilins, Kunitz domain peptides, Fynomers®, trispecific binding molecules and probodies.

4. Use of the nucleic acid molecule as defined in claim 1(I)(g) or the protein or peptide as defined in claim 1 in a sample obtained from a subject for diagnosing a tumor and/or for grading a tumor and/or for tumor prognosis and/or classifying tumor as a HLA-H low expression tumor or a HLA-H high expression tumor and/or for diagnosing an implantation failure.

5. A method for diagnosing a tumor comprising detecting the presence of the nucleic acid molecule as defined in claim 1(I)(g) and/or the protein or peptide as defined in claim 1 in a sample obtained from a subject, wherein the presence of the nucleic acid molecule as defined in claim 1(I)(g) and/or the protein as defined in claim 1 is indicative for a tumor in the subject.

6. A method for grading a tumor and/or for tumor prognosis comprising determining the level of the nucleic acid molecule of as defined in claim 1(I)(g) and/or the protein or peptide as defined in claim 1 in a sample obtained from a subject, wherein increased levels of the nucleic acid molecule as defined in claim 1(I)(g) and/or the protein or peptide as defined in claim 1 as compared to a control correlate with the a higher grade of the tumor and/or an adverse tumor prognosis.

7. Kit for diagnosing a tumor and/or for grading a tumor and/or for tumor prognosis, comprising
(a) means for the detection and/or quantification of the nucleic acid molecule as defined in claim 1(I)(g) and/or the protein or peptide as defined in claim 1 in a sample obtained from a subject, and
(b) instructions for using the kit.

8. A method for monitoring the non-efficacy of a tumor treatment in a subject having a tumor comprising (a) determining the amount of the nucleic acid molecule as defined claim 1(I)(g) and/or the protein or peptide as defined in claim 1 in a sample obtained from a subject before the start of the treatment; and (b) determining the amount of the nucleic acid molecule as defined in claim 1(I)(g) and/or the protein or peptide as defined in claim 1 in a sample obtained from a subject at one or more times after the start of the treatment, wherein an increased amount in b) as compared to a) is indicative for the non-efficacy of a tumor treatment and/or a decreased amount in b) as compared to a) is indicative for the efficacy of a tumor treatment.

9. A method for monitoring the non-efficacy of a immunosuppressive therapy in a subject requiring such a therapy comprising (a) determining the amount of the nucleic acid molecule as defined in claim 1(I)(g) and/or the protein or peptide as defined in claim 1 in a sample obtained from a subject before the start of the therapy; and (b) determining the amount of the nucleic acid molecule as defined in claim 1(I)(g) and/or the protein or peptide as defined in claim 1 in a sample obtained from a subject at one or more times after the start of the therapy, wherein a decreased amount in b) as compared to a) is indicative for the non-efficacy of a immunosuppressive therapy and/or an increased amount in b) as compared to a) is indicative for the efficacy of a immunosuppressive therapy.

10. The nucleic acid molecule, the vector, the host cell, and/or the protein or peptide, or combinations thereof of claim 1, the binding molecule, preferably the inhibitor of claim 2 or 3, the use of claim 4, the method of claim 5, 6 or 8, or the kit of claim 7, wherein the tumor is cancer.

11. The nucleic acid molecule, the vector, the host cell, and/or the protein or peptide, or combinations thereof, the binding molecule, preferably the inhibitor, the use, the method or the kit of claim 10, wherein the cancer is selected from the group consisting of breast cancer, ovarian cancer, endometrial cancer, vaginal cancer, vulva cancer, bladder cancer, salivary gland cancer, pancreatic cancer, thyroid cancer, kidney cancer, lung cancer, cancer concerning the upper gastrointestinal tract, colon cancer, colorectal cancer, prostate cancer, squamous-cell carcinoma of the head and neck, cervical cancer, glioblastomas, malignant ascites, lymphomas and leukemias.

12. The nucleic acid molecule, the vector, the host cell, and/or the protein or peptide, or combinations thereof, the binding molecule, preferably the inhibitor, the use, the method or the kit of claim 10, wherein the cancer is bladder cancer or a gynecologic cancer.

13. The nucleic acid molecule, the vector, the host cell, and/or the protein or peptide, or combinations thereof, the binding molecule, preferably the inhibitor, the use, the method or the kit of claim 10, wherein the cancer is breast cancer or ovarian cancer.

14. The nucleic acid molecule, the vector, the host cell, and/or the protein or peptide, or combinations thereof, the binding molecule, preferably the inhibitor, the use, the method or the kit of any preceding claim, wherein the sample is a body fluid or a tissue sample from an organ.


SEQUENCE LISTING (54 sequences)

SEQ ID NO: 1 - protein, 226 aa, Homo sapiens
  HLA-H
PQTPRMVLMAPRTLLLLLSGALALTLTQTWARSHSMRYFYTTMSRPGRGEPRFISVGYVDDTQFVRFDSDDASPREEPRAPWMEREGPEYWDRNTQICKAQAQTERENLRIALRYYNQSEGGSHTMQVMYGCDVGPDGRFLRGYEQHAYDGKDYIALNEDLRSWTAADMAAQITKRKWEAARRAEQLRAYLEGEFVEWLRRYLENGKETLQRADPPQDTYDPPPHL

SEQ ID NO: 2 - DNA, 1704 bp, Homo sapiens
  HLA-H
agtttctcttcttctcacaacctgcgacgggtccttcttccttgatactcacgaagcggacacagttctcattcccactaggtgtcgggtttctagagaagccaatcggtgccgccgcggtcccggttctaaagtccccacgcacccaccgggactcagattctccccagacgccgaggatggtgctcatggcgccccgaaccctcctcctgctgctctcaggggccctggccctgaccctgacccagacctgggcgcgctcccactccatgaggtatttctacaccaccatgtcccggcccggccgcggggagccccgcttcatctccgtcggctacgtggacgatacgcagttcgtgcggttcgacagcgacgacgcgagtccgagagaggagccgcgggcgccgtggatggagcgggaggggccagagtattgggaccggaacacacagatctgcaaggcccaggcacagactgaacgagagaacctgcggatcgcgctccgctactacaaccagagcgagggcggttctcacaccatgcaggtgatgtatggctgcgacgtggggcccgacgggcgcttcctccgcgggtatgaacagcacgcctacgacggcaaggattacatcgccctgaacgaggacctgcgctcctggaccgcggcggacatggcagctcagatcaccaagcgcaagtgggaggcggcccgtcgggcggagcagctgagagcctacctggagggcgagttcgtggagtggctccgcagatacctggagaacgggaaggagacgctgcagcgcgcggaccccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccgagcccctcaccctgagatgggagccatcttcccagcccaacgtccccatcgtgggcatcgttgctggcctggttctacttgtagctgtggtcactggagctgtggtcgctgctgtaatgtggaggaagaagagctcagatagaaaaggagggagctactctcaggctgcaacagcaacagtgcccagggctctgatgtgtctctcacggcttgaaagtgtgagacagctgccttgtgtgggactgagaggcaagagttgttcctgccttccctttgtgacttgaagaaccctgactttctttctacaaaggcacctgaatgtgtctgtgttcctgtaggcataatgtgtggaggaggggagaccaacccaccctcatgtccaccatgaccctcttccccacgctgatctgtgttccctccccaatcatctttcctgttccagagaggaggggctgagatgtctccatctttttctcaactttatgtgcactgagctgtaacttcttacttccctcttaaaattagaatctgagtaaacatttactttttcaaattcttgccatgagaggttgatgacttaattaaaggagaagattcctaaaatttgagagacaaaataaatggaacacatgagaaccttccag

SEQ ID NO: 3 - DNA, 270 bp, Homo sapiens
  HLA-H alpha 1 domain (Exon 2)
gctcccactccatgaggtatttctacaccaccatgtcccggcccggccgcggggagccccgcttcatctccgtcggctacgtggacgatacgcagttcgtgcggttcgacagcgacgacgcgagtccgagagaggagccgcgggcgccgtggatggagcgggaggggccagagtattgggaccggaacacacagatctgcaaggcccaggcacagactgaacgagagaacctgcggatcgcgctccgctactacaaccagagcgagggcg

SEQ ID NO: 4 - DNA, 277 bp, Homo sapiens
  HLA-H alpha 3 domain (Exon 4)
accccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccgagcccctcaccctgagatggg

SEQ ID NO: 5 - protein, 96 aa, Homo sapiens
  HLA-H alpha 1 domain
LTQTWARSHSMRYFYTTMSRPGRGEPRFISVGYVDDTQFVRFDSDDASPREEPRAPWMEREGPEYWDRNTQICKAQAQTERENLRIALRYYNQSEG

SEQ ID NO: 6 - protein, 13 aa, Homo sapiens
  HLA-H alpha 3 domain
DPPQDTYDPPPHL

SEQ ID NO: 7 - protein, 5 aa, Artificial Sequence
  GS linker
GGGGS

SEQ ID NO: 8 - protein, 4 aa, Artificial Sequence
  Redox-active site
CGPC

SEQ ID NO: 9 - DNA, 20 bp, Artificial Sequence
  Forward Primer HLA-G Ex2/3
ccgaaccctcttcctgctgc

SEQ ID NO: 10 - DNA, 20 bp, Artificial Sequence
  Probe HLA-G Ex2/3
cgagacctgggcgggctccc

SEQ ID NO: 11 - DNA, 20 bp, Artificial Sequence
  Reverse Primer HLA-G Ex2/3
gcgctgaaatacctcatgga

SEQ ID NO: 12 - DNA, 19 bp, Artificial Sequence
  Forward Primer HLA-H Ex2/3
gagagaacctgcggatcgc

SEQ ID NO: 13 - DNA, 24 bp, Artificial Sequence
  Probe HLA-H Ex2/3
agcgagggcggttctcacaccatg

SEQ ID NO: 14 - DNA, 19 bp, Artificial Sequence
  Reverse Primer HLA-H Ex2/3
ccacgtcgcagccatacat

SEQ ID NO: 15 - DNA, 18 bp, Artificial Sequence
  Forward Primer KRT5
cgccacttaccgcaagct

SEQ ID NO: 16 - DNA, 24 bp, Artificial Sequence
  Probe KRT5
tggagggcgaggaatgcagactca

SEQ ID NO: 17 - DNA, 25 bp, Artificial Sequence
  Reverse Primer KRT5
acagagatgttgactggtccaactc

SEQ ID NO: 18 - DNA, 26 bp, Artificial Sequence
  Forward Primer KRT20
gcgactacagtgcatattacagacaa

SEQ ID NO: 19 - DNA, 32 bp, Artificial Sequence
  Probe KRT20
ttgaagagctgcgaagtcagattaaggatgct

SEQ ID NO: 20 - DNA, 21 bp, Artificial Sequence
  Reverse Primer KRT20
cacaccgagcattttgcagtt

SEQ ID NO: 21 - DNA, 17 bp, Artificial Sequence
  Forward Primer PD-1
ggccagcccctgaagga

SEQ ID NO: 22 - DNA, 23 bp, Artificial Sequence
  Probe PD-1
acccctcagccgtgcctgtgttc

SEQ ID NO: 23 - DNA, 22 bp, Artificial Sequence
  Reverse Primer PD-1
ggaaatccagctccccatagtc

SEQ ID NO: 24 - DNA, 30 bp, Artificial Sequence
  Forward Primer PDL1
caaagtgatacacatttggaggagacgtaa

SEQ ID NO: 25 - DNA, 23 bp, Artificial Sequence
  Probe PDL1
tggcatccaagatacaaactcaa

SEQ ID NO: 26 - DNA, 24 bp, Artificial Sequence
  Reverse Primer PDL1
ttgaagatcagaagttccaatgct

SEQ ID NO: 27 - DNA, 24 bp, Artificial Sequence
  Forward Primer FGFR1
aattcaaacctgaccacagaattg

SEQ ID NO: 28 - DNA, 25 bp, Artificial Sequence
  Probe FGFR1
aggctacaaggtccgttatgccacc

SEQ ID NO: 29 - DNA, 24 bp, Artificial Sequence
  Reverse Primer FGFR1
caccacagagtccattatgatgct

SEQ ID NO: 30 - DNA, 19 bp, Artificial Sequence
  Forward Primer FGFR2
aagcaggagcatcgcattg

SEQ ID NO: 31 - DNA, 28 bp, Artificial Sequence
  Probe FGFR2
aggctacaaggtacgaaaccagcactgg

SEQ ID NO: 32 - DNA, 21 bp, Artificial Sequence
  Reverse Primer FGFR2
cagatgggaccacactttcca

SEQ ID NO: 33 - DNA, 19 bp, Artificial Sequence
  Forward Primer FGFR3
agcgcgtactgtgccactt

SEQ ID NO: 34 - DNA, 23 bp, Artificial Sequence
  Probe FGFR3
agtgtgcgggtgacagacgctcc

SEQ ID NO: 35 - DNA, 20 bp, Artificial Sequence
  Reverse Primer FGFR3
ctccccgtcttcgtcatctc

SEQ ID NO: 36 - DNA, 20 bp, Artificial Sequence
  Forward Primer FGFR4
ggatggacaggcctttcatg

SEQ ID NO: 37 - DNA, 21 bp, Artificial Sequence
  Probe FGFR4
cattggaggcattcggctgcg

SEQ ID NO: 38 - DNA, 21 bp, Artificial Sequence
  Reverse Primer FGFR4
cacgagactccagtgctgatg

SEQ ID NO: 39 - DNA, 20 bp, Artificial Sequence
  Forward Primer CALM2
gagcgagctgagtggttgtg

SEQ ID NO: 40 - DNA, 22 bp, Artificial Sequence
  Probe CALM2
tcgcgtctcggaaaccggtagc

SEQ ID NO: 41 - DNA, 21 bp, Artificial Sequence
  Reverse Primer CALM2
agtcagttggtcagccatgct

SEQ ID NO: 42 - DNA, 24 bp, Artificial Sequence
  Forward Primer ESR1
gccaaattgtgtttgatggattaa

SEQ ID NO: 43 - DNA, 19 bp, Artificial Sequence
  Probe ESR1
atgcccttttgccgatgca

SEQ ID NO: 44 - DNA, 27 bp, Artificial Sequence
  Reverse Primer ESR1
gacaaaaccgagtcacatcagtaatag

SEQ ID NO: 45 - DNA, 20 bp, Artificial Sequence
  Forward Primer ERBB1
tctggacgtgccagtgtgaa

SEQ ID NO: 46 - DNA, 22 bp, Artificial Sequence
  Probe ERBB1
aggccaagtccgcagaagccct

SEQ ID NO: 47 - DNA, 20 bp, Artificial Sequence
  Reverse PrimerERBB1
cctgctccctgaggacacat

SEQ ID NO: 48 - DNA, 21 bp, Artificial Sequence
  Forward Primer MMP28
tgcctaacgctagccttcaga

SEQ ID NO: 49 - DNA, 35 bp, Artificial Sequence
  Probe MMP28
cctcctaatgaagaaaggaaacctaatcagacccc

SEQ ID NO: 50 - DNA, 20 bp, Artificial Sequence
  Reverse Primer MMP28
ggcttaagtccccagcttga

SEQ ID NO: 51 - DNA, 26 bp, Artificial Sequence
  Forward Primer HPRT1
gggtgtttattcctcatggactaatt

SEQ ID NO: 52 - DNA, 27 bp, Artificial Sequence
  Probe HPRT1
tggacaggactgaacgtcttgctcgag

SEQ ID NO: 53 - DNA, 20 bp, Artificial Sequence
  Reverse Primer HPRT1
ggcctcccatctccttcatc

SEQ ID NO: 54 - protein, 221 aa, Homo sapiens
  HLA-H
MVLMAPRTLLLLLSGALALTLTQTWARSHSMRYFYTTMSRPGRGEPRFISVGYVDDTQFVRFDSDDASPREEPRAPWMEREGPEYWDRNTQICKAQAQTERENLRIALRYYNQSEGGSHTMQVMYGCDVGPDGRFLRGYEQHAYDGKDYIALNEDLRSWTAADMAAQITKRKWEAARRAEQLRAYLEGEFVEWLRRYLENGKETLQRADPPQDTYDPPPHL